SEQUENCE LISTING (30 sequences)

SEQ ID NO: 1 - protein, 1738 aa, Homo sapiens
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 2 - protein, 173 aa, Homo sapiens
INKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTY

SEQ ID NO: 3 - protein, 57 aa, Homo sapiens
HCVVNDTKPGNVTWNSLAIPDTHCSPELEEGYQCPPGFKCMDLEDLGLSRQELGYSG

SEQ ID NO: 4 - protein, 74 aa, Homo sapiens
FNEIGTSIFTVYEAASQEGWVFLMYRAIDSFPRWRSYFYFITLIFFLAWLVKNVFIAVIIETFAEIRVQFQQMW

SEQ ID NO: 5 - skipped

SEQ ID NO: 6 - protein, 115 aa, Homo sapiens
ACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKIS

SEQ ID NO: 7 - protein, 11 aa, Homo sapiens
PALEDFVYKIF

SEQ ID NO: 8 - protein, 105 aa, Homo sapiens
GPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELD

SEQ ID NO: 9 - skipped

SEQ ID NO: 10 - protein, 10 aa, Homo sapiens
EDLKKLKQLK

SEQ ID NO: 11 - protein, 158 aa, Homo sapiens
EHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLV

SEQ ID NO: 12 - protein, 12 aa, Homo sapiens
PQMRKVVRELFS

SEQ ID NO: 13 - protein, 58 aa, Homo sapiens
KLAKCNDPNIIRREDCNGIFRINVSVSKNLNLKLRPGEKKPGFWVPRVWANPRNFNFD

SEQ ID NO: 14 - protein, 64 aa, Homo sapiens
NVGNAMLALFEVLSLKGWVEVRDVIIHRVGPIHGIYIHVFVFLGCMIGLTLFVGVVIANFNENK

SEQ ID NO: 15 - skipped

SEQ ID NO: 16 - protein, 33 aa, Homo sapiens
GTALLTVDQRRWEDLKSRLKIAQPLHLPPRPDN

SEQ ID NO: 17 - protein, 123 aa, Homo sapiens
DGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHV

SEQ ID NO: 18 - protein, 15 aa, Homo sapiens
TLKMLLLTVVVSMYK

SEQ ID NO: 19 - protein, 19 aa, Homo sapiens
FGTVKYGENINRHANFSSA

SEQ ID NO: 20 - protein, 23 aa, Homo sapiens
KAITVLFRIVTGEDWNKIMHDCM

SEQ ID NO: 21 - skipped

SEQ ID NO: 22 - protein, 20 aa, Homo sapiens
VQPPFCTPDEFTYWATDCGN

SEQ ID NO: 23 - protein, 142 aa, Homo sapiens
LLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQ

SEQ ID NO: 24 - DNA, 25 bp, synthetic construct
  misc_feature 1..25: note = NALCN & NALCN flx PCR primer
  source 1..25: mol_type = other DNA
attgtccgtgagattgctcatcacc

SEQ ID NO: 25 - DNA, 25 bp, synthetic construct
  misc_feature 1..25: note = NALCN & NALCN Flx PCR primer
  source 1..25: mol_type = other DNA
gcaccagctatatgtccctctcacg

SEQ ID NO: 26 - DNA, 27 bp, synthetic construct
  misc_feature 1..27: note = NALCN Flx PCR primer
  source 1..27: mol_type = other DNA
ggaaaatgaccacttcctagcagaagc

SEQ ID NO: 27 - DNA, 17 bp, synthetic construct
  misc_feature 1..17: note = NALCN sense Quantitative PCR primer
  source 1..17: mol_type = other DNA
gccctcagcccccaaac

SEQ ID NO: 28 - DNA, 20 bp, synthetic construct
  misc_feature 1..20: note = NALCN anti-sense Quantitative PCR primer
  source 1..20: mol_type = other DNA
ggaagctgtgtctggcatgg

SEQ ID NO: 29 - DNA, 21 bp, synthetic construct
  misc_feature 1..21: note = GAPDH sense Quantitative PCR primer
  source 1..21: mol_type = other DNA
aggtcggtgtgaacggatttg

SEQ ID NO: 30 - DNA, 23 bp, synthetic construct
  misc_feature 1..23: note = GAPDH anti-sense Quantitative PCR primer
  source 1..23: mol_type = other DNA
tgtagaccatgtagttgaggtca

(19) United States
(12) Patent Application Publication
(10) Pub. No.: US 2026/0284115 A1
(43) Pub. Date: Sep. 24, 2026

(54) THERAPEUTICS AND METHODS

(71) Applicant: CAMBRIDGE ENTERPRISE LIMITED, Cambridge (GB)

(72) Inventors: Richard GILBERTSON, Cambridge (GB); Eric RAHRMANN, Cambridge (GB)

(21) Appl. No.: 18/874,336

(22) PCT Filed: Jun. 16, 2023
(86) PCT No.: PCT/GB2023/051590

§ 371 Date: Dec. 12, 2024

(30) Foreign Application Priority Data
Jun. 17, 2022 (GB) 2208948.6

(51) Int. Cl. A61K 35/22; A61K 35/37; A61P 13/12; C07K 14/705; C12N 5/071

(52) U.S. Cl. CPC A61K 35/22; A61K 35/37; A61P 13/12; C07K 14/705; C12N 5/068; C12N 5/0687; C12N 2510/00

(57) ABSTRACT

The invention relates to a modified stem cell-like cell for use in organ regeneration or repair wherein the modified stem cell-like cell exhibits reduced ion flow through sodium leak channel (NALCN). The invention also relates to an ion channel modulator for use in organ regeneration or repair. The invention further relates to associated methods, for example, a method of repairing or regenerating an organ.

### FIELD OF INVENTION

The invention relates to a modified stem cell-like cell for use in organ regeneration or repair. The invention also relates to an ion channel modulator for use in organ regeneration or repair. The invention further relates to associated methods, for example, a method of repairing or regenerating an organ.

### BACKGROUND

Regenerative medicine is a field which seeks to repair or replace damaged or diseased human cells or tissues to allow normal function to be restored. Organ and tissue loss and damage can occur in a variety of ways, including through disease, injury or congenital abnormalities, for example. The field of regenerative medicine is generally considered an interdisciplinary field applying developments in both engineering and biological sciences to promote the regeneration of damaged or diseased human cells or tissues. Approaches generally include tissue engineering and cellular therapies, for example using stem cells to regenerate damaged tissue, and there are a number of regenerative medicine therapies available. However, there are problems associated with the development of regenerative medicine. In relation to cellular therapies, one challenge includes how to effectively and consistently deliver such therapeutics, for example, stem cells, to the tissue or organs such as the kidney in need of regeneration or repair. One such example is the kidney whereby human patients afflicted with fibrosis in the kidneys have further progression of kidney disease and damage leading to higher rates of mortality. Identifying and treating patients at high risk of kidney disease or early stages of kidney disease to prevent further disease progression or even possibly reverse tissue dames would be hugely impactful.

An object of the present invention is to develop therapeutics for use in regenerating or repairing organs and/or tissues, i.e. for regenerative medicine.

### SUMMARY OF THE INVENTION

The present inventors have identified a single ion channel, NALCN, as a key regulator of epithelial cell shedding. The inventors have shown that stem cell-like epithelial cells in which Nalcn is deleted or treated with chemical entities to reduce the ion flow through NALCN cause the cells to mobilise to distinct tissues such as the lungs, liver, pancreas, kidneys and peritoneum and form normal structures in these organs, including kidney glomeruli and tubules and lung bronchioles. The transcriptomes of these circulating cells in tumour and non-tumour-bearing mice were indistinguishable and closely related to those of human circulating tumour cells (CTCs). The inventors have shown that NALCN regulates cell shedding from solid tissues independent of cancer. As such, cells modified to exhibit reduced ion flow through NALCN may be useful in repairing or regenerating organs or tissues.

The present invention therefore provides a modified stem cell-like cell for use in organ regeneration or repair, wherein the modified stem cell-like cell exhibits reduced ion flow through sodium leak channel (NALCN).

Also provided is an ion channel modulator for use in organ regeneration or repair, wherein the ion channel modulator reduces ion flow through sodium leak channel (NALCN).

The ion channel modulator may target NALCN and/or one of the proteins associated with NALCN, wherein the proteins associated with NALCN are selected from: GPCR (M3 muscarinic receptor M3R, TACR1, CaSR), UNC80, UNC79, FAM155A (NLF1A), Fam155B, SLO2.1.

The ion channel modulator may target the pore turret domains, voltage sensing domains or linker domains of NALCN.

The organ to be regenerated may be selected from: thymus, adrenal gland, thyroid gland, intestine, lungs, heart, liver, blood vessels, germ cells, nervous system, eye tissues, hair cells, kidney and bladder, skin, hair follicles, pancreas, bone, and cartilage.

The modified stem cell-like cell or ion channel modulator may be for use in the treatment of chronic kidney disease, for example glomerulonephritis and/or renal failure, lung disease, for example COPD, or liver fibrosing diseases.

The reduction in ion flow through NALCN may be transient.

Also provided is a pharmaceutical composition comprising the modified stem cell-like cell or ion channel modulator.

The present invention also provides a method of repairing or regenerating an organ in a subject, comprising administering a therapeutically effective amount of the modified stem cell-like cell, ion channel modulator or pharmaceutical composition described herein to the subject.

The method may further comprise obtaining stem cell-like cells from the subject to be treated.

The method may further comprise modifying the stem cell-like cells such that the stem cell-like cells exhibit reduced ion flow through NALCN.

The stem cell-like cells may be modified by:
contacting a stem cell-like cell with an ion channel modulator, wherein the ion channel modulator inhibits ion flow through NALCN; and/or
introducing a mutation in and/or deleting Nalcn and/or reducing expression of Nalcn in the stem cell-like cell, wherein the mutation and/or deletion and/or reduced expression results in reduced ion flow through NALCN.

The modified stem cell-like cell may be an epithelial stem cell.

The modified stem cell-like cell may:
i) comprise a mutation in Nalcn which reduces ion flow through NALCN; or
ii) have a knockout of Nalcn;
iii) exhibit reduced expression of Nalcn; or
iv) have been treated with an ion channel modulator to reduce ion flow through NALCN.

The reduced ion flow through NALCN may be transient.

Also provided is a method of preparing a modified stem cell-like cell the method comprising:
i) contacting a stem cell-like cell with an ion channel modulator, wherein the ion channel modulator inhibits ion flow through NALCN; or
ii) introducing a mutation in and/or deleting Nalcn and/or reducing the expression of Nalcn in a stem cell-like cell, wherein the mutation and/or deletion and/or reduced expression results in reduced ion flow through NALCN.

The stem cell-like cell may be an epithelial stem cell.

The contacting may be performed in vitro or ex vivo.

The method may further comprises expanding the modified stem cell-like cells.

Also provided is a method of increasing the level of solid tissue cell shedding of stem cell-like cells comprising introducing a mutation in and/or deleting Nalcn and/or reducing the expression of Nalcn in a stem cell-like cell, wherein the mutation and/or deletion and/or reduced expression results in reduced ion flow through NALCN.

Also provided is a method of increasing the level of solid tissue cell shedding of stem cell-like cells comprising contacting a cell with an ion channel modulator wherein said ion channel modulator is capable of reducing ion flow through NALCN.

The cell may be contacted in vitro, ex vivo or in vivo.

The solid tissue may be epithelial tissue.

Also provided is a kit comprising a modified stem cell-like cell, wherein the modified stem cell-like cell exhibits reduced ion flow through NALCN and optionally instructions for use.

The kit may further comprise components selected from one or more of; a NALCN modulator, cell culture media, buffers, excipients, vessels for cell culture

### FIGURES

FIG. 1. NALCN loss-of-function increases shedding of circulating non-tumour cells. (a) circulating non-tumour cells (ntCZCs) identified in non-tumour bearing mice (bar=median). The graph also shows the ntCZCs identified in mice with wildtype NALCN (labelled Nalcn^{+/+}) when the mice are exposed to Gadolinium chloride. (b) UMAP of 201,183 single cell RNA sequencing profiles of PBMCs, tCZCs and ntCZCs as well as cells derived from the indicated normal and malignant mouse tissues. (c) Co-immunofluorescence of ntCZCs and PBMCs in peripheral blood smears of P1^{R}Nalcn^{Flx/Flx} mice (ZsGreen [ZSG], scale bar=10 μm). (d) Direct ZSG-immunofluorescence photomicrographs of ZSG⁺ cells in lung and kidney (scale=50 μm) and enumerated in lung (e) and kidney (f). (g) Organ heat map of total numbers of ZSG⁺ cell clusters per mouse identified in organs of recipient mice injected with P1^{R}Nalcn^{+/+} ntCZCs or P1^{R}Nalcn^{Flx/Flx} ntCZCs. (h) Co-immunofluorescence of P1^{R}Nalcn^{Flx/Flx} ntCZCs (arrows) incorporated into the kidneys of recipient mice (arrows indicated ZSG⁺ cells, scale bar=50 μm). (i) Confocal laser scanning microscope image of P1^{R}Nalcn^{Flx/Flx} CZCs incorporated into the renal cortex of recipient mice (scale bar=100 μm). In all panels *=p<0.05; ***=p<0.0005, Mann-Whitney.

FIG. 2. NALCN loss-of-function circulating non-tumour cells (ntCZCs) resemble human and mouse CTCs and embed in distant organs. (a) Heatmap reporting geneset enrichment analysis in the UMAP clusters identified in FIG. 1b. Test Genesets were derived from 2,086 different tissue and cell types including bulk RNAseq of mouse normal tissues and tumours, huCTC signatures, and mouse and human intestinal stem and mature cell signatures (see Methods). (b) ZSG immunohistochemistry of aged Pdx1^{R}Nalcn^{+/+} (top left) and Pdx1^{R}Nalcn^{Flx/Flx} (bottom left) mouse lung bronchioles (scale=100 μm). Right, the number of ZSG+ cells/bronchiole in the lungs of Pdx1^{R}Nalcn^{+/+} (n=7) and Pdx1^{R}Nalcn^{Flx/Flx} (n=15; **, p<0.005 Mann-Whitney). (c) Two-photon direct ZSG+ cell clusters detected in entire lung section of a Pdx1^{R}Nalcn^{Flx/Flx} mouse. (d) Exemplar co-immunofluorescence of tail vein injected P1^{R}Nalcn^{Flx/Flx} ntCZCs (arrows) incorporated into the organs of recipient mice (arrows indicated ZSG⁺ cells, scale bar=50 μm).

FIG. 3. Engrafted ZSG⁺ ntCZCs express kidney markers. (a) UMAP of 166,878 SCS profiles of ntCZCs, engrafted ZSG⁺ ntCZCs into kidneys of immunocompromised mice (NOD scid-gamma—NSG), control NSG kidney cortex and medulla. (b-f) Feature plots of genes expressed in the kidney projected on the UMAP in (a): Bowman's capsule (Ly6a), glomerulus (Clic5), proximal tubule (Lrp2), loop of Henle (Clcnkb) and distal tubule (Calb1).

FIG. 4. ZSG⁺ cells are present in regions of kidney damage in vivo. (a) Schematic of the acute kidney damage model. (b) Measure of ZSG⁺ circulating cells (ntCZC) in Villin1-CreERT2; Rosa26-ZSGreen (V1Z) mice at Day −5 kidney damage induction. (c) Plot indicates weight loss which is associated with disease progression and stage. (d) Exemplar immunofluorescent images of kidneys from control (on left) and folic acid-treated (right) animals. Yellow indicates PDGFRb expression, a marker of inflammation and fibrosis in kidney disease. Light blue is the epithelial marker E-cadherin. (e) Bright field image (4×) of the kidney indicating the presence of ZSG⁺ cells. (f-g) Exemplar immunofluorescent images of kidneys from folic acid-treated mice depicting the presence of ZSG⁺ cells in structures that appear to be renal tubules. Yellow indicates PDGFRb expression, a marker of inflammation and fibrosis in kidney disease. Light blue is the epithelial marker E-cadherin. Red is the vessel marker CD31. (h) Scatter plot indicating the number of ZSG⁺ cells sorted per 10⁶ cells isolated from the kidneys of two folic acid-treated animals.

FIG. 5. Schematic of cellular therapeutic applications of ntCZCs.

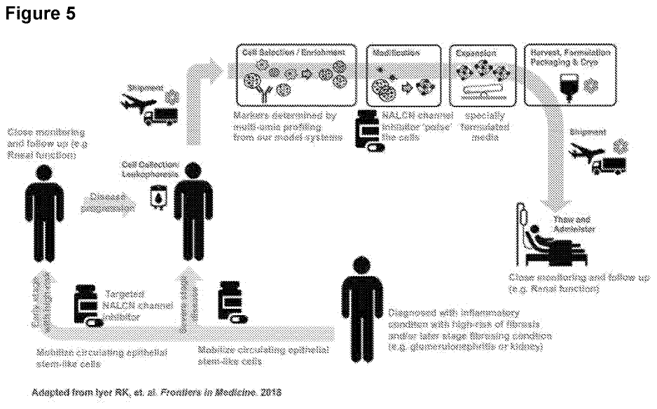

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

Ion channels are crucial components of cellular excitability and are involved in many diseases. The present inventors have herein demonstrated that NALCN plays a crucial role in epithelial stem-cell like cell shedding and non-malignant cell metastasis. NALCN is a nonselective monovalent cation channel which is a sole member of a distinct branch of voltage-gated sodium and calcium channels that regulates the resting membrane potential and excitability of neurons. NALCN is expressed most abundantly in the nervous system and conducts a persistent sodium leak current that contributes to tonic neuronal excitability. The sequence of NALCN is known and may comprise the sequence provided in ENSG00000102452 (ensemble), 259232 (NCBI Entrez Gene), 19082 (HGNC), Q8IZF0 (UniProtKB/Swiss-Prot), or 611549 (OMIM). In one embodiment the sequence of NALCN comprises SEQ ID NO.1. There are multiple splice variants of NALCN and the present invention extends to these variants. NALCN forms a polypeptide chain of 24 transmembrane helices (TM) that form four homologous functional repeats, also referred to as α-subunits, connected by intracellular linkers. Each functional repeat comprises voltage sensing domain, pore helices and ion selectivity filter.

It has been shown that loss of function of NALCN contributes to epithelial stem-cell like cell shedding in a non-cancerous model. Such cells mobilise to distinct tissues such as the lungs, liver, kidneys, pancreas and peritoneum and form normal structures in these organs. Therefore, such modified cells and ion channel modulators which reduce the ion flow through NALCN can be used in regenerative medicine, i.e. to repair or regenerate an organ or tissue.

As such, a first aspect the invention relates to a modified stem cell-like cell for use in organ regeneration or repair, wherein the modified stem cell-like cell exhibits reduced ion flow through sodium leak channel NALCN, referred to as NALCN herein.

A second aspect of the invention relates to an ion channel modulator for use in organ regeneration or repair, wherein the ion channel modulator reduces ion flow through sodium leak channel NALCN.

The present inventors have shown that by inhibiting ion flow through NALCN, whether that be by mutation or knockout of Nalcn, or treatment with an NALCN modulator, shedding of stem cell-like cells of epithelial origin increases. These cells mobilise to distinct tissues and/or organs and form normal structures in those organs. The present inventors have therefore identified that the inhibition or reduction of ion flow through NALCN can be used to repair or regenerate damaged or diseased organs and/or tissues.

### Organ Repair and Regeneration

As will be appreciated by the skilled person, the phrase organ regeneration or repair as used herein is used to mean a damaged or defective organ or tissue is repaired. The repair may not be complete repair, but an improvement. For example, the present invention may result in an improvement in the functioning of an organ or tissue compared to an untreated individual. For example, the present invention may result in an improvement in the functioning of an organ or tissue by 5%, 10%, 15%, 20%, 25%, 30%, 35%, 40%, 45%, 50%, 55%, 60%, 65%, 70%, 75%, 80%, 85%, 90%, 95% or 100% compared to an untreated individual.

The present invention may be used to repair or regenerate one or more of the following organs or organ systems: thymus, adrenal gland, thyroid gland, intestine, lungs, heart, liver, blood vessels, germ cells, nervous system, eye tissues, hair cells, kidney and bladder, skin, hair follicles, pancreas, bone, and cartilage. In embodiments, the present invention may be used to repair or regenerate one or more of the following organs or organ systems: intestine, lungs, liver, kidney, bladder, pancreas. In embodiments, the present invention may be used to repair or regenerate the kidney, liver, pancreas, intestine, lungs or bladder. In embodiments, the present invention may be used to repair or regenerate the kidney. As such, the present invention may be used to treat chronic kidney disease, for example glomerulonephritis and/or renal failure. The present invention may further be used to treat lung disease, for example COPD, or liver fibrosing diseases.

### Cells

In the present invention, a modified stem cell-like cell is used for repair or regeneration of an organ or tissue. The skilled person will readily appreciate that stem cells may be categorised from other cells in a number of ways. Properties of stem cells can be illustrated in vitro using, for example, clonogenic assays in which single cells are assessed for their ability to differentiate and self-renew. Stem cells may also be identified by the distinctive set of surface markers present on the surface of stem cells.

The term “stem cell-like cell” as used herein means that the cells share properties with stem cells and this term includes stem cells. In particular, the cells are progenitor and highly-plastic cells i.e. have the capability to form various cell types. The stem cell like-cells are capable of further differentiation into, or are not yet, a terminally differentiated cell type and/or somatic cell. Transcriptomic profiling by the inventors demonstrated that the stem cell-like cells have transcriptomic profiles very similar to stem cells such as gastric stem cells, gastric isthmus cells, small intestine stem cells, duodenal transit amplying cells and human circulating tumour cells which have demonstrated high-levels of plasticity to regenerate entire tumours. Epithelial stem cell-like cells of the present invention have been shown to exhibit enrichment of the following epithelial (e.g Cdh1, Epcam, Krt8, Krt18, Krt80, Krt222) and stem cell/epithelial-to-mesenchymal transition/metastasis initiating cell/circulating tumour cell markers (e.g. Zeb2, Tgfb1, Tgfbr1, Cd36, Cd44, L1cam, Fn1, Lgals3, Hba-a1, Hba-a2, Hbb-bs, Hbb-bt). Enrichment of these markers can be observed either transcriptionally and/or at the protein level, using histological/antibody-based approaches. Such markers may therefore be used to identify the stem cell-like cells of the invention.

Therefore, in embodiments, the stem cell-like cells of the invention exhibit enrichment of one or more of the following markers: Cdh1, Epcam, Krt8, Krt18, Krt80, Krt222, Zeb2, Tgfb1, Tgfbr1, Cd36, Cd44, L1cam, Fn1, Lgals3, Hba-a1, Hba-a2, Hbb-bs, Hbb-bt, Cacybp, Ceacam1.

The stem cell-like cell may be of epithelial origin. As will be appreciated by the skilled person, “of epithelial origin” means that the cell is epithelium-derived i.e. is a stem cell-like cell of the epithelium. The present inventors have shown that the stem cell-like cells of epithelial origin exhibit enrichment of the following markers: CDX2, CDH1 and KRT80 by immunofluorescence analysis. In addition, transcriptomic data has shown the enrichment of epithelial-specific markers Cdh1, Epcam, Cacybp, Ceacam1, Krt8, Krt18, Krt80 and Krt222. Enrichment of these markers can be observed either transcriptionally and/or at the protein level, using histological/antibody-based approaches. Such markers may therefore be used to identify the epithelial stem cell-like cells of the invention.

Therefore, in embodiments, the stem cell-like cells of epithelial origin exhibit enrichment of one or more of the following markers: CDX2, CDH1, KRT80, Epcam, Cacybp, Ceacam1, Cdh1, Krt8, Krt18, Krt80 and Krt222.

The inventors have shown herein that the modified stem cell-like cells can mobilise to distinct tissues and/or organs and form normal structures in those organs. In particular it is shown herein that the cells can mobilise to the kidney and subsequently begin to express markers of mature kidney cells including Ly6a, Clic5, Calb1, Clcnkb, Lrp2.

In a related aspect of the invention, the stem cell-like cell may be a stem cell. In another aspect of the invention, the stem cell is a stem cell of epithelial origin and/or an epithelial stem cell. The stem cell according to the invention is a cell that is capable of further differentiation into, or is not yet, a terminally differentiated cell type and/or somatic cell.

The stem cell may be any cell type selected from the following: pluripotent stem cells, multipotent stem cells, oligopotent stem cells or unipotent stem cells. It will be apparent to the skilled person that although pluripotent stem cells are directly derived from totipotent cells from an embryo, pluripotent cells may also be artificially derived from a non-pluripotent cell such as a somatic cell. Such artificially derived pluripotent stem cells are termed induced pluripotent stem cells. The stem cells of the present invention need not be derived from a human embryo.

### Reduced Ion Flow Through NALCN

It has been shown herein that the loss of function of NALCN contributes to an increase in the shedding of circulating non-tumour cells (ntCZC) and seeding of these cells in remote organs. As such by modulating the activity of NALCN it may be possible to increase or promote organ or tissue repair and/or regeneration.

In the present invention, the cells are modified such that the modified stem cell-like cell exhibits reduced ion flow through NALCN. Ion flow refers to the maximum number of ion molecules able to flow through NALCN in a given unit of time. According to embodiments of the invention, reducing ion flow involves preventing or impairing the passage of ion molecules through NALCN so that fewer ion molecules flow through NALCN in the same given unit of time. As NALCN is an ion channel responsible for the resting Na⁺ permeability of cells the activity of NALCN may be assessed using a variety of techniques. Activity may be assessed by whole-cell electrophysiology, a fluorescence assay, a membrane potential sensing dye, and/or an ion flux assay.

The ion channel modulator of the present invention modulates NALCN such the ion flow through NALCN is reduced. Ion channel modulators may be categorised as ion channel blockers or ion channel openers. Ion channel blockers are generally antagonistic compounds which act to prevent the response that is normally provided by the opening of the channel. In a preferred embodiment, the ion channel modulator is an ion channel blocker or a compound that is antagonistic to NALCN. As set out above, ion flow refers to the maximum number of ion molecules able to flow through NALCN in a given unit of time. According to embodiments of the invention, reducing ion flow involves preventing or impairing the passage of ion molecules through NALCN so that fewer ion molecules flow through NALCN in the same given unit of time. As NALCN is an ion channel responsible for the resting Na⁺ permeability of cells the activity of NALCN may be assessed using a variety of techniques. Activity may be assessed by whole-cell electrophysiology, a fluorescence assay, a membrane potential sensing dye, and/or an ion flux assay.

### Ion Channel Modulator

The ion channel modulator of the present invention may comprise a small molecule, an antibody or fragment thereof, a peptide, a synthetic peptide, a transcriptional modulator such as triplex-forming oligonucleotides, synthetic polyamides, zinc finger proteins, or post-transcriptional modulator such as RNAi, siRNA, miRNA.

The ion channel modulator may modulate the activity of NALCN by directly acting on the protein NALCN. For example, as a small molecule, an antibody a peptide, or a synthetic peptide. Alternatively, or in addition, the ion channel modulator may modulate the activity of NALCN by altering the expression level of the Nalcn gene or the NALCN protein. For example, as a transcriptional or post-transcriptional modulator.

Where the ion channel modulator acts on the NALCN protein, the modulation may be achieved by binding one or more of the α-subunits of NALCN. The modulation may be achieved by the modulator disrupting the protein-protein interactions between α-subunit and auxiliary subunits of NALCN or disrupting interactions between NALCN and protein(s) associated with NALCN. The modulation may be achieved by the modulator interfering with or enhancing the movement of the voltage sensor or other elements of the gating machinery.

As discussed above the ion channel modulator reduces ion flow through the NALCN. The ion channel modulator may reduce the ion flow through NALCN by targeting NALCN directly. The ion channel modulator may reduce the ion flow through NALCN by targeting one of the proteins associated with NALCN. NALCN protein forms a channelosome complex within the cell membrane. The channelosome includes various proteins associated with NALCN including G-protein-coupled receptors, UNC-79, UN-80, SLO2.1, FAM155A, FAM155B, NCA localization factor-1, and src family tyrosine kinases. As such one of these proteins that is associated with NALCN may be targeted by the ion channel modulator. The proteins associated with NALCN which may be targeted by the ion channel modulator are selected from: GPCR (M3 muscarinic receptor M3R, TACR1, CaSR), UNC80, UNC79, FAM155A (NLF1A), Fam155B, SLO2.1, src family tyrosine kinases.

In embodiments, the ion channel modulator may comprise, for example, gadolinium chloride, verapamil, high levels of exogenous Ca²⁺, 2-aminoethoxydiphenyl borate, nifedipine, nimodipine, flunarizine, ethoxzo amide, N-benzhydryl quinuclidine compounds, and/or L703606.

The NALCN protein comprises multiple domains and, as such, the ion channel modulator may target one or more of these domains in order to reduce ion flow through NALCN. The ion channel modulator may target one of more of the pore turret domains, voltage sensing domains or linker domains of NALCN. The linker domains may be linker domains that extend either extracellularly or intracellularly. The mutation may be in one or more of the domains comprising any one of the amino acid sequences set out in SEQ ID NO. 2 to 23. The domains of NALCN and their sequences are set out in the following table:

TABLE 1

| NALCN | |
|---|---|
| Domain | Amino acid sequence |
| Full length | MLKRKQSSRVEAQPVTDFGPDESLSDN |
| NALCN (SEQ | ADILWINKPWVHSLLRICAIISVISVCMNTP |
| ID NO. 1) | MTFEHYPPLQYVTFTLDTLLMFLYTAEMIA |
| | KMHIRGIVKGDSSYVKDRWCVFDGFMVF |
| | CLWVSLVLQVFEIADIVDQMSPWGMLRIPR |
| | PLIMIRAFRIYFRFELPRTRITNILKRSGEQIWS |
| | VSIFLLFFLLLYGILGVQMFGTFTYHCVVNDT |
| | KPGNVTWNSLAIPDTHCSPELEEGYQCPPG |
| | FKCMDLEDLGLSRQELGYSGFNEIGTSIFTV |
| | YEAASQEGWVFLMYRAIDSFPRWRSYFYFIT |
| | LIFFLAWLVKNVFIAVIIETFAEIRVQFQQMW |
| | GSRSSTTSTATTQMFHEDAAGGWQLVAVDV |
| | NKPQGRAPACLQKMMRSSVFHMFILSMVTV |
| | DVIVAASNYYKGENFRRQYDEFYLAEVAFTVL |
| | FDLEALLKIWCLGFTGYISSSLHKFELLLVIGTT |
| | LHVYPDLYHSQFTYFQVLRVVRLIKISPALEDF |
| | VYKIFGPGKKLGSLVVFTASLLIVMSAISLQMFC |
| | FVEELDRFTTFPRAFMSMFQILTQEGWVDVMD |
| | QTLNAVGHMWAPVVAIYFILYHLFATLILLSLFV |
| | AVILDNLELDEDLKKLKQLKQSEANADTKEKLP |
| | LRLRIFEKFPNRPQMVKISKLPSDFTVPKIRESF |
| | MKQFIDRQQQDTCCLLRSLPTTSSSSCDHSKR |
| | SAIEDNKYIDQKLRKSVFSIRARNLLEKETAVT |
| | KILRACTRQRMLSGSFEGQPAKERSILSVQHH |
| | IRQERRSLRHGSNSQRISRGKSLETLTQDHSNT |
| | VRYRNAQREDSEIKMIQEKKEQAEMKRKVQEE |
| | ELRENHPYFDKPLFIVGREHRFRNFCRVVVRAR |
| | FNASKTDPVTGAVKNTKYHQLYDLLGLVTYLDW |
| | VMIIVTICSCISMMFESPFRRVMHAPTLQIAEYV |
| | FVIFMSIELNLKIMADGLFFTPTAVIRDFGGVMDIF |
| | IYLVSLIFLCWMPQNVPAESGAQLLMVLRCLRPL |
| | RIFKLVPQMRKVVRELFSGFKEIFLVSILLLTLMLV |
| | FASFGVQLFAGKLAKCNDPNIIRREDCNGIFRI |
| | NVSVSKNLNLKLRPGEKKPGFWVPRVWANPR |
| | NFNFDNVGNAMLALFEVLSLKGWVEVRDVIIHR |
| | VGPIHGIYIHVFVFLGCMIGLTLFVGVVIANFNEN |
| | KGTALLTVDQRRWEDLKSRLKIAQPLHLPPRPDN |
| | DGFRAKMYDITQHPFFKRTIALLVLAQSVLLSVKW |
| | DVEDPVTVPLATMSVVFTFIFVLEVTMKIIAMSPA |
| | GFWQSRRNRYDLLVTSLGVVWVVLHFALLNAYT |
| | YMMGACVIVFRFFSICGKHVTLKMLLLTVVVSM |
| | YKSFFIIVGMFLLLLCYAFAGVVLFGTVKYGENIN |
| | RHANFSSAGKAITVLFRIVTGEDWNKIMHDCMVQ |
| | PPFCTPDEFTYWATDCGNYAGALMYFCSFYVIIAY |
| | IMLNLLVAIIVENFSLFYSTEEDQLLSYNDLRHFQIIW |
| | NMVDDKREGVIPTFRVKFLLRLLRGRLEVDLDKD |
| | KLLFKHMCYEMERLHNGGDVTFHDVLSMLSYRSV |
| | DIRKSLQLEELLAREQLEYTIEEEVAKQTIRMWLK |
| | KCLKRIRAKQQQSCSIIHSLRESQQQELSRFLNPPS |
| | IETTQPSEDTNANSQDNSMQPETSSQQQLLSPTL |
| | SDRGGSRQDAADAGKPQRKFGQWRLPSAP |
| | KPISHSVSSVNLRFGGRTTMKSVVCKMNPMTDAAS |
| | CGSEVKKWWTRQLTVESDESGDDLLDI |
| Voltage | INKPWVHSLLRICAIISVISVCMNTPMTFEHYPPLQYV |
| sensing | TFTLDTLLMFLYTAEMIAKMHIRGIVKGDSSYVKDRW |
| domain 1 | CVFDGFMVFCLWVSLVLQVFEIADIVDQMSPWGMLR |
| (SEQ ID NO. | IPRPLIMIRAFRIYFRFELPRTRITNILKRSGEQIWS |
| 2) | VSIFLLFFLLLYGILGVQMFGTFTY |
| Extracellular | HCVVNDTKPGNVTWNSLAIPDTHCSPELEEGYQCP |
| linker 1 (SEQ | PGFKCMDLEDLGLSRQELGYSG |
| ID NO. 3) | |
| Pore turret | FNEIGTSIFTVYEAASQEGWVFLMYRAIDSFPRWRSYF |
| domain 1 | YFITLIFFLAWLVKNVFIAVIIETFAEIRVQFQQMW |
| (SEQ ID NO. | |
| 4) | |
| Selective | QEG |
| filter (SEQ ID | |
| NO. 5) | |
| Voltage | ACLQKMMRSSVFHMFILSMVTVDVIVAASNYYKGEN |
| sensing | FRRQYDEFYLAEVAFTVLFDLEALLKIWCLGFTGYIS |
| domain 2 | SSLHKFELLLVIGTTLHVYPDLYHSQFTYFQVLRVVRL |
| (SEQ ID NO. | IKIS |
| 6) | |
| S4-S5 linker | PALEDFVYKIF |
| (SEQ ID | |
| NO. 7) | |
| Pore turret | GPGKKLGSLVVFTASLLIVMSAISLQMFCFVEELDRFTTF |
| domain 2 | PRAFMSMFQILTQEGWVDVMDQTLNAVGHMWAPVVA |
| (SEQ ID NO. | IYFILYHLFATLILLSLFVAVILDNLELD |
| 8) | |
| Selective | QEG |
| filter (SEQ ID | |
| NO. 9) | |
| DII-DIII linker | EDLKKLKQLK |
| (SEQ ID NO. | |
| 10) | |
| Voltage | EHRFRNFCRVVVRARFNASKTDPVTGAVKNTKYH |
| sensing | QLYDLLGLVTYLDWVMIIVTICSCISMMFESPFRRVMH |
| domain 3 | APTLQIAEYVFVIFMSIELNLKIMADGLFFTPTAVIRDFG |
| (SEQ ID | GVMDIFIYLVSLIFLCWMPQNVPAESGAQLLMVLRCL |
| NO. 11) | RPLRIFKLV |
| S4-S5 linker | PQMRKVVRELFS |
| (SEQ ID NO. | |
| 12) | |
| Extracellular | KLAKCNDPNIIRREDCNGIFRINVSVSKNLNLKLRPGEK |
| linker 2 (SEQ | KPGFWVPRVWANPRNFNFD |
| ID NO. 13) | |
| Pore turret | NVGNAMLALFEVLSLKGWVEVRDVIIHRVGPIHGIYIH |
| domain 3 | VFVFLGCMIGLTLFVGVVIANFNENK |
| (SEQ ID NO. | |
| 14) | |
| Selective | LKG |
| filter (SEQ ID | |
| NO. 15) | |
| DIII-DIV | GTALLTVDQRRWEDLKSRLKIAQPLHLPPRPDN |
| linker (SEQ | |
| ID NO. 16) | |
| Voltage | DGFRAKMYDITQHPFFKRTIALLVLAQSVLLSVKWDV |
| sensing | EDPVTVPLATMSVVFTFIFVLEVTMKIIAMSPAGFWQ |
| domain 4 | SRRNRYDLLVTSLGVVWVVLHFALLNAYTYMMGAC |
| (SEQ ID NO. | VIVFRFFSICGKHV |
| 17) | |
| S4-S5 linker | TLKMLLLTVVVSMYK |
| (SEQ ID NO. | |
| 18) | |
| Extracellular | FGTVKYGENINRHANFSSA |
| linker 3 (SEQ | |
| ID NO. 19) | |
| Pore turret | KAITVLFRIVTGEDWNKIMHDCM |
| domain 4 | |
| (SEQ ID | |
| NO. 20) | |
| Selective | GED |
| filter (SEQ ID | |
| NO. 21) | |
| Extracellular | VQPPFCTPDEFTYWATDCGN |
| linker 4 (SEQ | |
| ID NO. 22) | |
| C-terminal | LLSYNDLRHFQIIWNMVDDKREGVIPTFRVKFLLRL |
| domain (SEQ | LRGRLEVDLDKDKLLFKHMCYEMERLHNGGDVTF |
| ID NO. 23) | HDVLSMLSYRSVDIRKSLQLEELLAREQLEYTIEEE |
| | VAKQTIRMWLKKCLKRIRAKQQQSCSIIHSLRESQQ |

The ion channel modulator may also interact with specific residues within NALCN. For example the ion channel modulator may interact with one or more of the amino acid residues of NALCN selected from, but not limited to: L588M, P573, R855, K1213, T71, P225, D1527, D416, C1348, R297, V1386, A1091, V1229, D134, T272, R43, A1157, V1036, M520, R1500, V320, V53, W1085, E1458, N1274, V1542, Y1300, R1174, H1523, F332, Q549, L999, F540, A1421, R1384, H569, A1435, M55, R1495, C245, F110, V510, C970, E454, V273, R1556, S174, S1068, V385, S384, A401, S902, R1495, A276, R1540, L517, R295, R382, H876, F300, R164, E257, R995, G1526, D291, V1239, E1552, N1475, M55, L1553, Y1349, E323, A1044, T1281, V1007, L253, L564, F1427, V949, Q279, T539, R159, K452, R1127, V1490, G555, E62, L1461, L942, R166, P65, D952, 1322, F154, K1163, L305, R152, W1085, R143, A1444, R989, R143, R1193, D1466, M520, V1285, S52, 151, E1518, E532, L1279, V1329, T57, A1378, S121, K498, R1094, V120, A88, A401, L1548, G1303, M150, D1277, E432, L1442, P1082, T1165, G1316, R1273, E128, E906, F1311, R1481, T204, T552, F389, D1527, P908, A1166, 1577, G954, G1013, P65, E1016, N1070, S980, A1217, V1503, T1320, A223, A310, R1127, D1504, D1277, E128, K1491, Q553, V511, F1250, S1374, D211, T1149, D1099, M1425, M1003, P467, R43, L222, V400, M1244, A424, F1410, G193, H39, W219, F1018, R1193, K1069, V50, R1498, K1230, S403, S1264, R995, Q238, 11433, P66, L428, D1171, A1107, S1033, 11017, K1259, M986. It has been demonstrated by the present inventors that mutations at each of these positions can result in the closure of the NALCN pore. Without wishing to be bound by theory it is hypothesised that these amino acid residues may be involved in regulating the opening of the NALCN pore. As such by targeting one or more of these residues it may be possible to reduce ion flow through NALCN.

The reduction in ion flow through NALCN may be temporary (i.e. transient) or permanent. In preferred embodiments, the reduction in ion flow is transient. In such embodiments, the cells may be treated with the ion channel modifier to transiently reduce ion flow through NALCN. Such a transient reduction would allow trafficking of stem-cell like cells to remote organs or tissues in need of regeneration or repair. Once the stem-cell like cells had integrated in and formed normal structures in these organs or tissues, normal ion flow through NALCN may resume.

The ion channel modulator of the present invention may not completely prevent ion flow through NALCN, but simply reduce ion flow through NALCN. For example, the ion channel modulator may reduce the ion flow through NALCN by 10%, 20%, 30%, 40%, 50%, 60%, 70%, 75%, 80%, 85%, 90%, 95%, 96%, 97%, 98%, 99% or 100% compared to ion flow through a non-treated cell/subject.

### Modification of Stem Cell-Like Cell

The present invention provides a modified stem cell-like cell for use in organ repair or regeneration. The modified stem cell-like cell may:
i) comprise a mutation in Nalcn which reduces ion flow through NALCN; or
ii) have a knockout of Nalcn;
iii) exhibit reduced expression of Nalcn; or
iv) have been treated with an ion channel modulator to reduce ion flow through NALCN.

As will be appreciated by the skilled person, a knockout of Nalcn means that the cell does not have a Nalcn gene or the Nalcn gene has been disrupted (as the gene has been removed or inactivated by genetic engineering, for example). Methods of producing Nalcn knockouts will be well known by the skilled person and include, for example, random mutagenesis and selection, for example using radiation or chemical mutagenesis, homologous recombination-based approaches, or newer technologies such as CRISPR.

It is shown herein that deletion of Nalcn can cause upregulation of one or more genes selected from Mmp10, Mmp19, Mmp7, Mmp9, Fn1, Zeb1, Fstl1, Sparc, Sfrp4, Cdh6, and/or Timp3. As such, a cell with a Nalcn deletion or mutation may be characterised by enrichment for one or more of Mmp10, Mmp19, Mmp7, Mmp9, Fn1, Zeb1, Fstl1, Sparc, Sfrp4, Cdh6, and/or Timp3.

In embodiments in which the modified cell comprises a mutation in Nalcn, the mutation reduces ion flow through NALCN. As such, the mutation may be a loss of function mutation. The mutation may not completely prevent ion flow through NALCN, but simply reduce ion flow through NALCN, and as such may also be a reduction in function mutation. For example, the mutation may reduce the ion flow through NALCN by 10%, 20%, 30%, 40%, 50%, 60%, 70%, 75%, 80%, 85%, 90%, 95%, 96%, 97%, 98%, 99% or 100% compared to ion flow through a non-mutated Nalcn. A mutation may be introduced into Nalcn using any suitable method including site-directed mutagenesis zinc finger nucleases, transcription activator-like effector nucleases (TALENs) or CRISPR technology. The mutation may be introduced into Nalcn in vitro, ex vivo, or in vivo. Where the mutation is introduced in vitro or ex vivo the cells may be obtained from a subject prior to the step of introducing said mutation.

As set out above, the NALCN protein comprises multiple domains and, as such, the modified cell may comprise a mutation in one or more of these domains in order to reduce ion flow through NALCN. The mutation may be in one of more of the pore turret domains, voltage sensing domains or linker domains of NALCN. The linker domains may be linker domains that extend either extracellularly or intracellularly. The mutation may be in one or more of the domains comprising any one of the amino acid sequences set out in SEQ ID NO. 2 to 23. The domains of NALCN and their sequences are set out in Table 1. The modified stem cell-like cell may comprise a mutation in Nalcn which reduces ion flow through NALCN. The one or more of the mutations within NALCN may be present at positions selected from, but not limited to; L588M, P573, R855, K1213, T71, P225, D1527, D416, C1348, R297, V1386, A1091, V1229, D134, T272, R43, A1157, V1036, M520, R1500, V320, V53, W1085, E1458, N1274, V1542, Y1300, R1174, H1523, F332, Q549, L999, F540, A1421, R1384, H569, A1435, M55, R1495, C245, F110, V510, C970, E454, V273, R1556, S174, S1068, V385, S384, A401, S902, R1495, A276, R1540, L517, R295, R382, H876, F300, R164, E257, R995, G1526, D291, V1239, E1552, N1475, M55, L1553, Y1349, E323, A1044, T1281, V1007, L253, L564, F1427, V949, Q279, T539, R159, K452, R1127, V1490, G555, E62, L1461, L942, R166, P65, D952, 1322, F154, K1163, L305, R152, W1085, R143, A1444, R989, R143, R1193, D1466, M520, V1285, S52,151, E1518, E532, L1279, V1329, T57, A1378, S121, K498, R1094, V120, A88, A401, L1548, G1303, M150, D1277, E432, L1442, P1082, T1165, G1316, R1273, E128, E906, F1311, R1481, T204, T552, F389, D1527, P908, A1166, 1577, G954, G1013, P65, E1016, N1070, S980, A1217, V1503, T1320, A223, A310, R1127, D1504, D1277, E128, K1491, Q553, V511, F1250, S1374, D211, T1149, D1099, M1425, M1003, P467, R43, L222, V400, M1244, A424, F1410, G193, H39, W219, F1018, R1193, K1069, V50, R1498, K1230, S403, S1264, R995, Q238, 11433, P66, L428, D1171, A1107, S1033, 11017, K1259, M986. It has been demonstrated that mutations at each of these positions can result in the closure of the NALCN pore i.e. a reduction in the size of the NALCN pore and therefore a reduction in NALCN activity. It is hypothesised that these amino acid residues may be involved in regulating the opening of the NALCN pore as such mutations at one or more of these positions may result in a reduction in the pore diameter and therefore a reduction in NALCN activity (i.e. a reduction in ion flow through NALCN).

In embodiments in which the expression of Nalcn is reduced this may be by way of any suitable method, for example, RNAi, siRNA, zinc finger nucleases, transcription activator-like effector nucleases (TALENs) or CRISPR technology, suitable methods for reducing expression using these methods are known to the skilled person. The reduction in expression may be temporary (i.e. transient) or permanent. In preferred embodiments, the reduction in expression is transient. In such embodiments, the cells may be treated with RNAi or siRNA to temporarily reduce the expression of Nalcn. The expression of Nalcn may be modified using the ion channel modifier described above. In order to reduce the expression of Nalcn, the cell may be exposed to or contacted with RNAi, siRNA, CRISPR and/or an ion channel modulator, the step of exposing or contacting the cell may be performed in vitro, ex vivo or in vivo. Where the cell is exposed or contacted in vitro or ex vivo the cells may be obtained from a subject prior to the step of contacting or exposing said cells.

In embodiments in which the stem cell-like cells have been treated with an ion channel modulator to reduce ion flow through NALCN, the modulator may not completely prevent ion flow through NALCN, but simply reduce ion flow through NALCN. For example, the ion channel modulator may reduce the ion flow through NALCN by 10%, 20%, 30%, 40%, 50%, 60%, 70%, 75%, 80%, 85%, 90%, 95%, 96%, 97%, 98%, 99% or 100% compared to ion flow through a non-treated cell/subject.

### Pharmaceutical Compositions

In an aspect the invention relates to a pharmaceutical composition comprising a modified stem cell-like cell, wherein the modified stem cell-like cell exhibits reduced ion flow through NALCN.

In an aspect the invention relates to a pharmaceutical composition comprising an ion channel modulator, wherein the ion channel modulator reduces ion flow through NALCN.

The modified stem cell-like cell, ion channel modulator or the pharmaceutical composition may be administered by any suitable route for example any parenteral or enteral route. For example, any suitable route, includes but is not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation. Parenteral administration includes, for example, intravenous, intramuscular, intraarterial, intraperitoneal, intranasal, rectal, intravesical, intradermal, topical or subcutaneous administration. Preferably administration may be orally, sublingually, buccally, intravenously, intramuscularly, subcutaneously, rectally, or intranasally. Various oral dosage forms can be used, including such solid forms as tablets, capsules, liquids, granules and bulk powders. Tablets can be compressed, tablet triturates, enteric-coated, sugar-coated, film-coated, or multiple-compressed, containing suitable binders, lubricants, diluents, disintegrating agents, colouring agents, flavouring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, colouring agents and flavouring agents.

The modified stem cell-like cell, ion channel modulator, or the pharmaceutical composition may take the form of one or more dosage units.

In an embodiment the pharmaceutical composition of the invention may further comprise one or more additional active agents, pharmaceutically acceptable carrier, diluent or adjuvant. The pharmaceutically acceptable carrier, diluent or adjuvant may vary depending on the dosage form used. For example, various oral dosage forms can be used, including such solid forms as tablets, capsules, liquids, granules and bulk powders. Tablets can be compressed, tablet triturates, enteric-coated, sugar-coated, film-coated, or multiple-compressed, containing suitable binders, lubricants, diluents, disintegrating agents, colouring agents, flavouring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, colouring agents and flavouring agents. can be in the form of a liquid, e.g., a solution, emulsion or suspension. Liquid compositions, whether they are solutions, suspensions or other like form, can also include one or more of the following: sterile diluents such as water, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. The modified stem cell-like cell, ion channel modulator or pharmaceutical composition can be enclosed in an ampoule, a disposable syringe or a multiple-dose vial made of glass, plastic or other material.

An intravenous formulation of the modified stem cell-like cell, ion channel modulator, or pharmaceutical composition may be in the form of a sterile injectable aqueous or non-aqueous (e.g. oleaginous) solution or suspension. The sterile injectable preparation may also be in a sterile injectable solution or suspension in a non-toxic parenterally-acceptable diluent or solvent, for example, a solution in 1,3-butanediol. Among the acceptable vehicles and solvents that may be employed are water, phosphate buffer solution, Ringer's solution and isotonic sodium chloride solution. In addition, sterile, fixed oils may be employed as a solvent or suspending medium. For this purpose, any bland fixed oil may be employed, including synthetic mono- or diglycerides. In addition, fatty acids such as oleic acid may be used in the preparation of the intravenous formulation of the invention.

The modified stem cell-like cell, ion channel modulator or pharmaceutical composition can be prepared using methodology well known in the pharmaceutical art. For example, a composition intended to be administered by injection can be prepared by combining with water so as to form a solution. A surfactant can be added to facilitate the formation of a homogeneous solution or suspension.

### Method of Treatment/Organ Regeneration or Repair

In an aspect the invention relates to a method of organ regeneration or repair in a subject, comprising administering a therapeutically effective amount of modified stem cell-like cell or pharmaceutical composition comprising the modified stem cell-like cell to the subject, wherein the modified stem cell exhibits reduced ion flow through NALCN. Details of the modified stem cell-like cells are described above.

In an aspect the invention relates to a method of organ regeneration or repair in a subject, comprising administering a therapeutically effective amount of an ion channel modulator or pharmaceutical composition comprising the ion channel modulator to the subject, wherein the ion channel modulator reduces ion flow through NALCN. Details of the ion channel modulator are described above.

The present inventors have surprisingly shown that by inhibiting ion flow through NALCN, whether that be by mutation or knockout of Nalcn, or treatment with an NALCN modulator, shedding of stem cell-like cells of epithelial origin increases. These cells mobilise to distinct tissues and/or organs and form normal structures in those organs. The present inventors have therefore identified that the inhibition of ion flow through NALCN can be used to repair or regenerate damaged or diseased organs and/or tissues.

As will be appreciated by the skilled person, the phrase organ regeneration or repair as used herein is used to mean a damaged or defective organ or tissue is repaired. The repair may not be complete repair, but an improvement. For example, the present invention may result in an improvement in the functioning of an organ or tissue compared to an untreated individual. For example, the present invention may result in an improvement in the functioning of an organ or tissue by 5%, 10%, 15%, 20%, 25%, 30%, 35%, 40%, 45%, 50%, 55%, 60%, 65%, 70%, 75%, 80%, 85%, 90%, 95% or 100% compared to an untreated individual.

The present invention may be used to repair or regenerate one or more of the following organs or organ systems: thymus, adrenal gland, thyroid gland, intestine, lungs, heart, liver, blood vessels, germ cells, nervous system, eye tissues, hair cells, kidney and bladder, skin, hair follicles, pancreas, bone, and cartilage. In embodiments, the present invention may be used to repair or regenerate one or more of the following organs or organ systems: intestine, lungs, liver, kidney, bladder, pancreas. In embodiments, the present invention may be used to repair or regenerate the kidney, liver, pancreas, intestine, lungs or bladder. In embodiments, the present invention may be used to repair or regenerate the kidney. As such, the present invention may be used to treat chronic kidney disease, for example glomerulonephritis and/or renal failure. The present invention may further be used to treat lung disease, for example COPD, or liver fibrosing diseases.

In an embodiment, the modified cell, ion channel modulator or the pharmaceutical composition is for use in the treatment or prevention of organ and/or tissue loss and/or damage. The organ and/or tissue loss and/or damage may be caused by disease, injury and/or congenital abnormalities.

In an embodiment, the modified cell is for use in the treatment of organ and/or tissue loss and/or damage of one or more of the following organs or organ systems: thymus, adrenal gland, thyroid gland, intestine, lungs, heart, liver, blood vessels, germ cells, nervous system, eye tissues, hair cells, kidney and bladder, skin, hair follicles, pancreas, bone, and cartilage. In embodiments, the present invention may be used in the treatment of organ and/or tissue loss and/or damage of one or more of the following organs or organ systems: intestine, lungs, liver, kidney, bladder, pancreas. In embodiments, the present invention may be used in the treatment of organ and/or tissue loss and/or damage of the kidney, liver, pancreas, intestine, lungs or bladder. In embodiments, the present invention is in the treatment of organ and/or tissue loss and/or damage of the kidney. As such, the present invention may be used to treat chronic kidney disease, for example, glomerulonephritis and/or renal failure. The present invention may further be used to treat lung disease, for example COPD, or liver fibrosing diseases.

When the modified stem cell-like cell or ion channel modulator is used in the prevention of a disease, the modified stem cell-like cell or ion channel modulator may be administered prophylactically. When the modified stem cell-like cell or ion channel modulator is for use in the treatment of a disease, disease progression monitoring may be used to identify subjects in need of further therapy.

The modified stem cell-like cell, ion channel modulator or pharmaceutical composition may be used in combination with a further treatment. The further treatment may include other organ repair or regenerative therapies, for example tissue engineering and/or cellular therapies. Alternatively, or in addition, the further treatment may comprise a pharmaceutical treatment to improve the functioning of the organ or tissue.

The term “combination” as used herein, is meant to encompass the administration of the modified stem cell-like cell, ion channel modulator or pharmaceutical composition simultaneously, separately, or sequentially with administration of the further treatment.

The invention provides a method of organ repair or regeneration in a subject comprising administering a therapeutically effective amount of modified stem cell-like cells to the subject.

In some embodiments the stem cell-like cells are autologous. In these embodiments, the stem cell-like cells may be obtained from the subject to whom the modified stem-cell-like cells are to be administered.

Therefore, in embodiments, the method comprises:
i) obtaining stem cell-like cells from the subject to be treated.

In some embodiments the stem cell-like cells are allogenic. In these embodiments, the stem cell-like cells may be obtained from a suitable subject or donor person. Therefore, in embodiments, the method comprises:
i) obtaining stem cell-like cells from a suitable subject or donor person.

The skilled person will appreciate how such cells can be obtained from a subject, for example by way of a blood sample or tissue sample obtained from the subject. Methods of selecting stem-cell like cells from such a sample include for example size exclusion and antibody enrichment approaches (magnetic bead sorting or FACs) using defined cell surface markers for specific populations.

The stem cell-like cells may then be modified ex vivo such that the stem cell-like cell exhibits reduced ion flow through NALCN. The invention may therefore further comprise:
i) modifying the stem cell-like cells such that the stem cell-like cell exhibits reduced ion flow through NALCN.

Methods of modifying the stem cell-like cells to reduce ion flow through NALCN are discussed above.

The stem cell-like cells may be modified by:
a) contacting a stem cell-like cell with an ion channel modulator, wherein the ion channel modulator inhibits ion flow through NALCN; and/or
b) introducing a mutation in and/or deleting Nalcn and/or reducing expression of Nalcn in the stem cell-like cell, wherein the mutation and/or deletion and/or reduced expression results in reduced ion flow through NALCN.

The method may further comprise expanding the modified stem cell-like cells which have been modified to reduce ion flow through NALCN. The skilled person will appreciate how such cells may be expanded. The term “expanding” or “expansion of” stem cell-like cells, or any other cell type, as described herein describes an increase of cell number due to cell division. Culture media that allow the expansion of the stem cell-like cells are known to the skilled person and include, but are not limited to, IPS-Brew, PS-Brew XF, E8, StemFlex, mTeSR1, PluriSTEM, StemMACS, TeSRTM2, Corning NutriStem hPSC XF Medium, Essential 8 Medium (ThermoFisher Scientific), StemFit Basic02 (Ajinomoto Co. Inc), to name only a few. The temperature may influence whether the conditions are suitable for the expansion of the stem cell-like cells. Accordingly, the temperature of the culture medium may be about 30° C. to 50° C., about 35° C. to 40° C., about 36° C. to 38° C. or about 37° C. Oxygen levels may influence whether the conditions are suitable for the expansion of the stem cell-like cells. Accordingly, the oxygen percentage of the culture medium could range from normoxia (20% O₂) along a gradient of hypoxic conditions (0.5% O₂).

For the culture of stem cell-like cells, the culture surface such as a culture plate or culture vessel may be coated with an extracellular matrix (ECM) to provide support for the attached growth of cells. ECM is the outer cell surface matrix and mainly consists of proteins such as collagens, elastins and laminins. ECM is widely used in culturing of mammalian cells and is known to the skilled person. The non-limiting examples of ECM which can be used to coat the solid support or culturing surface, e.g. a culture plate, include Matrigel™, laminin, Poly-Lysine, a combination of polyornithine (PO)/fibronectin (FN)/laminin (lam), fibronectin (FN) and the like.

In embodiments, the expansion may include culturing the modified stem cell-like cells in culture medium. A medium comprises a mixture of nutrients required for the growth of cells of a certain type. A medium is usually prepared by adding supplements to a basal medium. Supplements refers to additional components which are not present in the basal media but may be required by the cell culture, including, but not limited to, proteins, lipids, amino acids, vitamins, hormones, cytokines, growth factors. The culture medium may contain specific growth factors, including, for example, one or more of insulin, growth factors like EGF, bFGF, N2, B27, wnt conditioned media, r-spondin, TGFbeta inhibitors. When more than one medium is used, medium exchange can be conducted at any time point during the culture by means well known to the skilled person.

A therapeutically effective amount of modified stem cell-like cells may then be administered to the subject.

The method of organ regeneration or repair in a subject may comprise different steps depending on the stage of the organ damage or the disease that is causing said organ damage. For example, wherein a subject is identified with a disease that is at an early stage but high risk, said subject may be initially treated with a targeted NALCN modulator. Said targeted NALCN modulator will allow mobilisation of circulating stem cell-like cells, preferably epithelial stem cell-like cells.

Wherein a subjected in treated with a NALCN modulator, close monitoring for disease progression can be used to monitor said subject to determine if follow up therapy is necessary. If the disease progresses further or to a severe stage, further therapy may be required comprising treatment with modified stem cell-like cell. The treatment with a modified stem cell-like cell may comprise the methods described above. In an embodiment treatment with a modified stem cell-like cell may comprise the steps of:
i) obtaining stem cell-like cells from said subject, a suitable subject or donor person,
ii) modifying the stem cell-like cells such that the stem cell-like cell exhibits reduced ion flow through NALCN:
   a. contacting a stem cell-like cell with an ion channel modulator, wherein the ion channel modulator inhibits ion flow through NALCN; and/or
   b. introducing a mutation in and/or deleting Nalcn and/or reducing expression of Nalcn in the stem cell-like cell, wherein the mutation and/or deletion and/or reduced expression results in reduced ion flow through NALCN.
iii) introducing the modified cells to said subject.

Where a subject is identified with a severe disease, for example wherein significant organ damage has already occurred said subject may be treated with a targeted NALCN modulator in combination with a modified stem cell-like cell wherein said modified stem cell-like cell has been prepared according to the methods described herein.

The invention also relates to the use of a modified stem cell-like cell or ion channel modulator for the manufacture of a medicament for organ regeneration or repair.

The subject to be treated may be any subject in need of repair or regeneration of an organ or tissue. In embodiments, the subject is a mammal, preferably a human.

The terms “treat”, “treatment” or “treating,” as used herein refer to administering a compound to a subject for prophylactic and/or therapeutic purposes. As explained herein, the modified cells of the present invention can be used to repair or regenerate an organ or tissue. Thus, in one embodiment, the modified cell may be used to prevent a decrease or further decrease in organ or tissue function and is administered prophylactically. As such the term “prevention” refers to preventing organ degeneration, in particular, the modified cell may be used to prevent organ degeneration (or further organ degeneration). For example, the modified cell may be used to treat a subject identified as being at a high risk of organ damage or degeneration or further damage or degeneration. The term “prevention” can refer to a reduction in the risk of organ degeneration or damage. The term “prophylactic or preventative treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, the terms “effective amount” and “therapeutically effective amount” refer to the quantity of the active therapeutic agent sufficient to yield a desired therapeutic response without undue adverse side effects such as toxicity, irritation, or allergic response. The specific “effective amount” will vary with such factors as the particular condition being treated, the physical condition of the patient, the type of subject being treated, the duration of the treatment, the nature of concurrent therapy (if any), and the specific formulations employed and the structure of the compounds or its derivatives.

### Other Methods

The present invention also provides a method of increasing the level of solid tissue cell shedding of stem-cell like cells comprising introducing a mutation in and/or deleting Nalcn and/or reducing the expression of Nalcn in a stem cell-like cell, wherein the mutation and/or deletion and/or reduced expression results in reduced ion flow through NALCN.

Mutation of and deletion of Nalcn and reducing the expression of Nalcn is described above.

In an aspect, the invention also relates to a method of increasing the level of solid tissue cell shedding of stem cell-like cells comprising contacting a cell with an ion channel modulator wherein said ion channel modulator reduces ion flow through NALCN.

It has been shown by the inventors that loss of function of NALCN markedly increases the levels of circulating stem cell-like cells. Therefore, by reducing ion flow through NALCN the level of circulating stem cell-like cells can be increased.

In an embodiment the cell is contacted in vitro, ex vivo or in vivo.

The ion channel modulators are described above.

The solid tissue may be epithelial tissue.

The present invention also provides a method of preparing a modified stem cell-like cell the method comprising:
i) contacting a stem cell-like cell with an ion channel modulator, wherein the ion channel modulator inhibits ion flow through NALCN; and/or
ii) introducing a mutation in and/or deleting Nalcn and/or reducing expression of Nalcn in the stem cell-like cell, wherein the mutation and/or deletion and/or reduced expression results in reduced ion flow through NALCN.

The ion channel modulators and stem cell-like cells are described above.

The method may further comprise expanding the modified stem cell-like cells which have been modified to reduce ion flow through NALCN. The skilled person will appreciate how such cells may be expanded. The expansion of cells, including suitable growth media and additional factors, is described above.

In embodiments, the method may comprise depleting terminally differentiated cells from the ex vivo culture. Suitable methods for depleting terminally differentiated cells include, for example, antibody enrichment approaches.

In embodiments, the stem cell-like cells to be modified may be obtained from a subject to be treated with the modified stem cell-like cells. Methods of obtaining such cells are described above.

### Kits

The present invention also provides a kit comprising a modified stem cell-like cell, wherein the modified stem cell-like cell exhibits reduced ion flow through NALCN and optionally instructions for use.

The kit may further comprise various other components. In an embodiment, the kit comprises a NALCN modulator, which may be any NALCN modulator as described herein. In an embodiment, the kit further comprises components for culturing of said modified stem cell-like cell. The cell culture components may be any culture media, buffer, excipient, vessel etc, as described herein.

The present invention also provides a kit comprising means to obtain a cell sample from a subject and a NALCN modulator and/or components to modify a cell to reduce ion flow through NALCN as described herein.

Unless otherwise defined herein, scientific and technical terms used in connection with the present disclosure shall have the meanings that are commonly understood by the skilled person. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless the context dictates otherwise, the descriptions and definitions of the features set out above are not limited to any particular aspect or embodiment of the invention and apply equally to all aspects and embodiments which are described.

The term “comprising” or “comprises” where used herein means including the component(s) specified but not to the exclusion of the presence of other components. The term “consisting essentially of” or “consists essentially of” means including the components specified but excluding other components except for materials present as impurities, unavoidable materials present as a result of processes used to provide the components and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

The invention is further illustrated in the following non-limiting examples.

### Methods

### Culture of Stomach Stem Cells

Gastric glands were isolated by perfusing mice with 30 mM EDTA/PBS, stomach removal and scraping pyloric mucosa into 10 mM EDTA/PBS at 4° C. Dissociated, filtered and resuspended cells were placed in Matrigel (354230, BD Bioscience) and culture medium: Advanced DMEM/F12 406 (31330038, ThermoFisher), B27 (12587010, ThermoFisher), N2 (A1370701, ThermoFisher), N-407 Acetylcysteine (A9165, Sigma-Aldrich), and 10 nM Gastrin (G9145, Sigma-Aldrich) containing growth factors (50 ng/ml EGF [Peprotech], 1 mg/ml R-spondin1 [120-38, PeproTech.], 100 ng/ml Noggin [250-38, Peprotech], 100 ng/ml FGF10 [100-26, Preprotech] and Wnt3A conditioned media). Gastric spheres were passaged by dispase (D4818, Sigma-Aldrich) digestion and dissociation into single cells (StemPro® Accutase, Life Technologies). Gadolinium (439770, Sigma-Aldrich) was diluted in the culture medium and overlaid on Matrigel embedded cells. Gastric glands isolated from Prom1^{CreERT2/Lacz}; Rosa26^{ZSG}; Nalcn^{Flx/Flx} mice were treated in vitro with 0.25 μM 4-hydroxytamoxifen to induce deletion of Nalcn. ZsGreen+ gastric organoids were harvested by FACs 72 hrs after induction and subjected to RNA isolation and transcriptomic profiling.

### Generation of NalcnFlx Allele

Mice were derived from targeted ES cells (UCDAVIS KOMP Repository Knockout Mouse Project clone EPD0383_5_C01). ES cells were screened using KOMP PCR strategies for Nalcntm1a(KOMP)Wstsi. ES cells were implanted into recipient C57/B16 mice in accordance with IACUC-SJ approved protocols. Wildtype Nalcn and Nalcn^{Flx} alleles were detected using standard polymerase chain reaction and primers (UCDAVIS KOMP Repository Knockout Mouse Project clone EPD0383_5_C01): 5″-ATTGTCCGTGAGATTGCTCATCACC-3″ (SEQ ID NO. 24) and 5″-GCACCAGCTATATGTCCCTCTCACG-3″ (SED ID NO. 25) for Nalcn wildtype or 5-GGAAAATGACCACTTCCTAGCAGAAGC-3″ (SEQ ID NO. 26) Nalcn^{Flx}.

Nalcn RNA expression was quantified by quantitative reverse transcriptase PCR and a Biorad CFX96 Touch Real-Time PCR Detection System with primers: Nalcn sense 5-GCCCTCAGCCCCCAAAC-3″ (SEQ ID NO. 27) (spans exon 43/44), Nalcn anti-sense 5″-GGAAGCTGTGTCTGGCATGG-3″ (SEQ ID NO. 28) (exon 44), Gapdh sense 5″-AGGTCGGTGTGAACGGATTTG-3″ (SEQ ID NO. 29) and Gapdh anti-sense 5-TGTAGACCATGTAGTTGAGGTCA-3 (SEQ ID NO. 30).

### Harvesting and Injection of Circulating ZsGreen+ Cells

Peripheral blood (500 μl-1 ml) was harvested from mice at autopsy into 10 μl of 0.5M EDTA, diluted in PBS and assessed by MACSQuant Analyzer (Miltenyi Biotech Inc.) for ZSgreen expression (525/50 nm [FITC] vs 614/50 nm [PI]). Cells for single cell RNA sequencing and tail vein injection were sorted using a BD FACSAria II Cell Sorter (BD Biosciences) excitation at 525/50 nm (FITC) vs 614/50 nm (PI). Non-tamoxifen induced mouse peripheral blood served as a negative control to set gate parameters. Twenty-five thousand ZSG⁺ cells were sorted and injected into recipient NOD SCID gamma mice (Charles River) and aged. For serial dilution assessment of tCZC metastasis initiation, tCZCs were isolated from donor tumour-bearing via FACS based on ZSGreen expression and placed into culture medium. Culture medium: Advanced DMEM/F12 (31330038, ThermoFisher), 2 mM L-glutamine (25030024, ThermoFisher), B27 (12587010, ThermoFisher), N2 (A1370701, ThermoFisher), containing growth factors (50 ng/ml EGF [Peprotech], 100 ng/ml bFGF [100-18c, Peprotech] and 1% FBS (10500064, ThermoFisher). Cells were grown at 37° C. in 5% CO₂. Recipient NOD SCID gamma mice (Charles River) were injected with either 10, 100, 1,000 or 10,000 tCZC cells via tail-vein injection and aged. Full autopsy and tissue harvesting were performed as described above.

Induction of Kidney Damage (Acute Model) and Mobilisation of ZSG+ ntCZCs In Vivo

Adult Villin1-CreERT2; Rosa26-ZSGreen; Nalcn+/+; Villin1-CreERT2; Rosa26-ZSGreen; NalcnFlx/+; Villin1-CreERT2; Rosa26-ZSGreen; NalcnFlx/Flx male and female mice were given 8 mg total of tamoxifen over two days to induce recombination of conditional alleles in the intestinal epithelium. Animals were aged for 2 months and then received a single intraperitoneal injection of Folic Acid (250 mg/kg) to induce kidney damage. Animals were monitored for weight loss daily for up to 7 days. At endpoint, kidneys were harvested and fixed in 1% paraformaldehyde for 24 hrs followed by a 10% sucrose solution and finally embedded into OCT for tissue sectioning and histological evaluation of fibrosis and the presence of ZSG+ cells in the kidneys.

### Histology

Hematoxylin and Eosin staining was performed using standard procedures (7221, 7111, Thermo 511 Fisher Scientific). Immunohistochemistry was performed using standard procedures and primary antibodies: Ki67 (IHC-00375, Bethyl, 1:1000), ZSGreen (632474, Clontech, 1:2000), Pan cytokeratin (AE1/AE3) (901-011-091620, BioCare Medical, 1:100), CK5 (ab52635, Abcam, 1:100), Vimentin (5741S, Cell Signalling Technology, 1:200), Cleaved Caspase 3 (9664, Cell Signaling Technology, 1:200), CD31 (77699, Cell Signaling Technology, 1:100), α-smooth muscle actin (ab5694, Abcam 1:500), CD45 (ab25386, Abcam, 5 μg/ml). Secondary antibodies were anti-rabbit Poly-HRP-IgG (included in kit) or Rabbit anti-Rat (A110-322A, Bethyl Laboratories, 1:250). Digital images of entire tissue sections were captured using the Leica Aperio AT2 digital scanner (40×, resolution 0.25 μM/pixel), viewed using the Leica Aperio Image Scope v12.3.2.8013 and quantified by HALO (Indica Labs) image analysis.

For immunofluorescence, tissue sections were incubated with primary antibodies: Rhodamine-labeled DBA (RL-1032, Vector Laboratories, 1:100), rhodamine-labeled UEA I (RL-1062, Vector Laboratories, 1:100), ZSGreen (TA180002, Origene, 1:1000), CD31 (102520, BioLegend, 1:100), CK7 (ab181598, Abcam, 1:200), CK20 (ab97511, Abcam, 1:200), E-cadherin (147308, BioLegend, 1;100)), PDGFRb (136005, BioLegend, 1:100), N-cadherin (13116, Cell Signalling Technology, 1:100), Icam1 (ab179707, Abcam, 1:100), Cdx2 (ab76541, Abcam, 1:100), Ttf1 (ab76013, Abcam, 1:100), Krt80 (16835-1-AP, ProteinTech, 1:100), Hba-a1 (ab92492, Abcam, 1:100), Lgals3 (ab209344, Abcam, 1:200), CD45 (ab10558, Abcam, 1:200). Secondary antibodies included Alexa 488, 594, 647 (A-11055, A-21207, A-31571, ThermoFisher, 1:500). Sections were counterstained (DAPI 4083; Cell Signaling, 1:10,000) and images captured using a Zeiss ImagerM2 and Apotome microscope or Zeiss Axioscan.Z1 (Zeiss) at 40× magnification and processed using ZEN2.3 (Zeiss) software.

Nalcn RNA expression was detected in FFPE sections using the Advanced Cell Diagnostics (ACD) RNAscope® 2.5 LS Reagent Kit-RED (ACD, 322150) and RNAscope 2.5 LS Mm Nalcn (ACD, 415168). Probe hybridisation and signal amplification was performed according to manufacturer's instructions. Fast Red detection of mouse Nalcn was performed was performed on the Bond Rx using the Bond Polymer Refine Red Detection Kit (Leica Biosystems, DS9390) according to manufacturer's protocol. Whole tissue sections were imaged on the Aperio AT2 (Leica Biosystems) and analysed as for immunohistochemistry using HALO (Indica Labs) imaging analysis software β-galactosidase staining was performed exactly as described.

Histological review, primary and metastatic tumour classification of was performed by performed by expert pathologists (Drs. Peter Vogel and Betania Mahler-Araujo) blinded to mouse genotype and clinical history. The numbers of ZSG⁺ cell clusters or metastases were counted in each organ in each mouse.

### Whole-Tissue Imaging

Kidneys were exsanguinated, perfused with PBS and 4% PFA by PBS washes and immersion reagent 1a [150 g ultra-pure water, 20 g Triton X-100 (10254583, Fisher Scientific), 10 g of 100% solution of N,N,N″,N″-tetrakis (2-hydroxypropyl)ethylenediamine (122262, Sigma), 20 g Urea (140750010, ACROS organics), 1 ml 5M NaCl] containing 10 μM DAPI (4083; Cell Signaling Technology) at 37° C. and 80 rpm. The solution was exchanged every 2 days until tissue was cleared. Cleared tissues were washed and immersed in 50% PBS/50% reagent 2 [15 g Ultra-pure water, 50 g sucrose (220900010, ACROS organics), 25 g urea (140750010, ACROS organics), 10 g 2,2,2-nitrilotriethanol (90279, Sigma)] for 6 hrs (room temperature, gentle shaking) followed by immersion in 100% reagent 2 (10 ml) for 1 day (room temperature). Tissues were mounted and scanned on a TCS SP5 confocal laser scanning microscope (Leica) at 10× objective for Dapi and endogenous expression of ZSGreen. Images were processed using Imaris x64v9.3.0 software (Oxford Instruments).

Serial two-photon tomography (STPT) imaging was performed on a TissueCyte 1000 instrument (TissueVision, MA USA) where a series of mosaic 2D images are taken of the tissue, followed by physical sectioning with a vibratome and a subsequent round of imaging. This continues in an automated fashion, generating 15 μm STPT sections which can be mounted on standard microscopy slides, imaged by Axioscan fluorescence scanning (Zeiss) for section identification and realignment. Fiducial agarose marker beads labelled with GFP are distributed throughout the embedding medium to help in the realignment of the samples for consequent use.

### Single Cell RNA Sequencing

Animals were perfused with PBS followed by 100 U/ml of Collagenase type IV in HBSS with Ca²⁺ and Mg²⁺ (Life Technologies) media containing 3 mM CaCl₂. Whole organs were dissected, dissociated and placed into 2 ml of appropriate dissociation buffer: lung and stomach were dissociated with 200 U/ml of Collagenase type IV (Sigma) and 100 μg/μl of DNAsel (Roche) in HBSS with Ca²⁺ and Mg²⁺ (Life Technologies) media containing 3 mM CaCl₂; liver was dissociated with Collagenase type I (100 U/ml), Dispase (2.4 U/ml) DNAsel (100 μg/ml) in HBSS with Ca²⁺ and Mg²⁺ (Life Technologies) media containing 3 mM CaCl₂; kidney was dissociated with Papain (20 U/ml) and DNAsel (100 mg/ml) in DMEM High glucose, 2 mM L-Glutamine (Life Technologies) with 1× Pen-605 Strep and 10% foetal bovine serum; uterus and epididymis were dissociated with Collagenase type I (100U/ml) and DNAsel (100 mg/ml) in in HBSS with Ca²⁺ and Mg²⁺ (Life Technologies) media containing 3 mM CaCl₂. Cells suspensions were filtered washed with HBSS without calcium and magnesium and centrifuged for 5 min at 300×g at 4° C. for 5 minutes.

Single-cell suspensions of solid tissues were multiplexed and labelled with Cell Hashing conjugates: anti-mouse hashtags from 0301-0315 (BioLegend) prior to sequencing. All nucleated cells and ZSG⁺ cells isolated from peripheral blood were not multiplexed but placed into 10× Genomics pipeline. Single-cell RNA-seq libraries were prepared using Chromium Single Cell 3′ Library & Gel Bead Kit v3, Chromium Chip B Kit and Chromium Single Cell 3′ Reagent Kits v3 User Guide (Manual Part CG000183 RevA; 10× Genomics). Cell suspensions were loaded on the Chromium instrument with the expectation of collecting gel-beads emulsions containing single cells. RNA from the barcoded cells for each sample was subsequently reverse-transcribed in a C1000 Touch Thermal cycler (Bio-Rad) and all subsequent steps to generate single-cell libraries were performed according to the manufacturer's protocol with no modifications (for most of the samples 12 cycles was used for cDNA amplification, 16 for samples with very low cell concentration). cDNA quality and quantity were measured with Agilent TapeStation 4200 (High Sensitivity 5000 ScreenTape) after which 25% of material was used for gene expression library preparation. Library quality was confirmed with Agilent TapeStation 4200 (High Sensitivity D1000 ScreenTape to evaluate library sizes) and Qubit 4.0 Fluorometer (ThermoFisher Qubit™ dsDNA HS Assay Kitto evaluate dsDNA quantity). Each sample was normalized and pooled in equal molar concentration. To confirm concentration pools were qPCRed using KAPA Library Quantification Kit on QuantStudio 6 Flex before sequencing. Pools were sequenced on Illumina NovaSeq6000 sequencer with following parameters: 28 bp, read 1; 8 bp, i7 index; and 91 bp, read 2.”

Raw RNA reads were processed with cellranger using mm10 from 10× as the reference genome to create filtered gene expression matrices. Cell barcodes detected by cellranger were used as input to CITESeq for hashtagged sequence data (solid organs) generating a counts matrix with cell barcodes and hashtag oligo sequences per cell. The HTODemux function from Seurat was then used to identify clusters and classify cells according to their barcodes including negative and doublet cells. Quality control metrics were generated using Scater followed by single cell object conversion to Seurat objects, merging of objects and then analyses run using the standard Seurat pipeline.

Single cell sequencing profiles of human CTCs (GSE75367; GSE74639; GSE60407; GSE67980; GSE114704; GSE144494) and 500 cells from Illumina 1 OX for human PBMC raw counts were merged in python (v3.7.3) using the pandas library. Only common genes between datasets were analysed. Seurat objects were created from PBMCs and CTCs. Following this step, data were analysed using the standard Seurat pipeline.

For direct comparison of human CTCs and mouse tCZC, 15,328 orthologues were identified and profiles processed through the standard Seurat workflow that includes a per-cell normalisation of each gene expression count. Enrichment of a haemoglobin gene expression was carried out in UCell and enrichment scores generated with a Mann-Whitney U statistic.

### Example 1: NALCN and Circulating Non-Cancer Cells

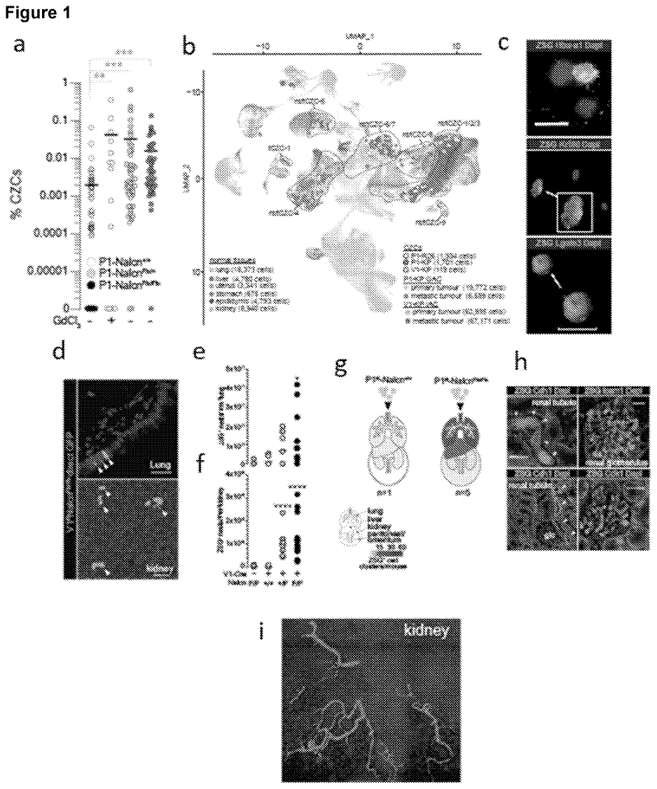

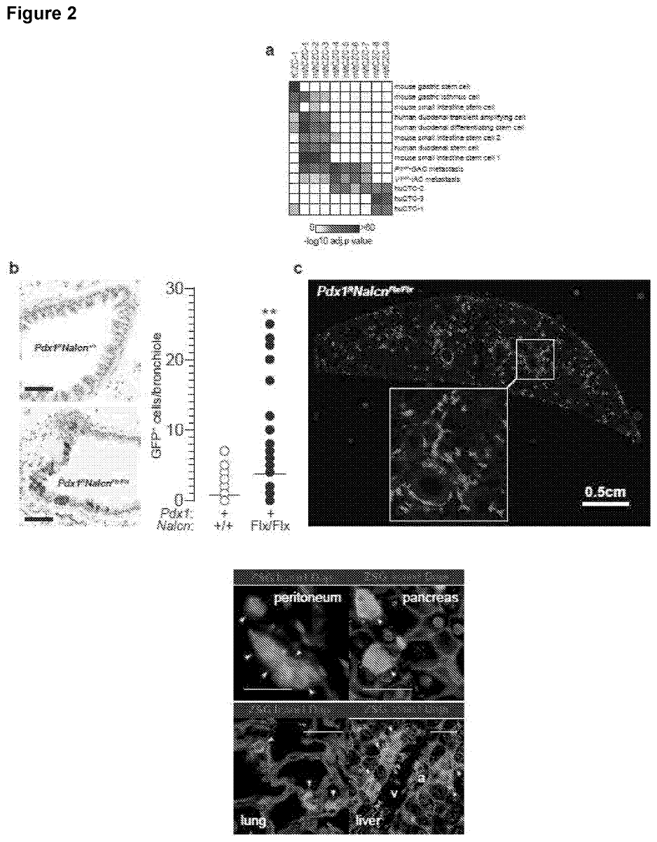

The inventors have shown that deletion of Nalcn from cells is associated with circulating tumour cell (CTC) shedding into peripheral blood and metastasis; however, disentangling this process from the complex cascade of tumourigenesis has proved challenging. Deletion of Nalcn from freshly isolated P1; Nalcn^{Flx/Flx} gastric stem cells that lacked oncogenic alleles, rapidly upregulated genes associated with invasion (e.g., Mmp7, Mmp9, Mmp10 and Mmp19) and gastric epithelial-mesenchymal transition (EMT) (e.g., Zeb1, Fstl1, Sparc, Sfrp4, Cdh6, and Timp3) (Table 2); suggesting NALCN might regulate cell shedding from solid tissues independent of transformation. To test this, the inventors looked for nucleated, ZSG-autofluorescent circulating cells (CZCs) in the peripheral blood of Prom1^{CreERT2/Lacz}; Rosa26^{ZSG}; Nalcn^{+/+} (P1^{R}Nalcn^{+/+} n=87), P1^{R}Nalcn^{+/Flx} (n=50) and P1^{R}Nalcn^{Flx/Flx} (n=37) mice that lacked oncogenic alleles and never developed tumours. Remarkably, deletion of Na/on increased the numbers of CZCs in these mice to levels similar to those observed in tumour-bearing animals (FIG. 1a). Similarly, blockade of NALCN with Gadolinium chloride (GdCl₃) also significantly increased CZCs in Prom1^{CreERT2/Lacz}; Rosa26^{ZSG}; Nalcn^{+/+} (P1RNalcn^{+/+} n=10) mice (FIG. 1a). Single cell RNA sequencing (SCS) profiles of CZCs isolated from non-tumour-bearing (ntCZCs) mice co-clustered with CZCs from tumour bearing animals (tCZC; FIG. 1b). The great majority of tCZCs and ntCZCs SCSs did not cluster with SCS profiles of primary IACs, GACs, or normal tissues but with SOS profiles of metastases (FIG. 1b). SOS profiles of both tCZCs and ntCZCs matched those of huCTCs, and similar to human CTCs, expressed genes associated with epithelial, stem and progenitor cells (Table 3); although tCZCs were relatively more enriched for metastasis and invasion-associated genesets (FIG. 2a; Table 4). Co-immunofluorescence of blood smears confirmed that both ntCZCs and tCZCs share markers of huCTCs, including Hba-a1, Krt80 and Lgals3 (FIG. 1c). Further, transcriptomic analysis demonstrated enrichment of various epithelial, stem and progenitor cell markers (Table 3).

TABLE 2
Upregulated genes in gastric stem cells with deletion of Nalcn

| Gene | log2fold change | Adjusted p value |
|---|---|---|
| Mmp10 | 1.18323743 | 0.009513763 |
| Mmp19 | 2.822634333 | 0.038511359 |
| Mmp7 | 0.590344189 | 0.040734017 |
| Mmp9 | 4.785724906 | 0.015460478 |
| Fn1 | 0.518777467 | 0.022334527 |
| Zeb1 | 0.553679318 | 0.033168147 |
| Fstl1 | 2.243475264 | 0.046253864 |
| Sparc | 1.813734028 | 0.039747804 |
| Sfrp4 | 0.933819677 | 0.038275845 |
| Cdh6 | 0.61829413 | 0.044006673 |
| Timp3 | 0.598178181 | 0.032579363 |

TABLE 3
Upregulated genes associated with CZCs

| Gene | Average log fold change | Adjusted p value |
|---|---|---|
| L1cam | 0.895770782 | 4.76E−08 |
| Krt80 | 0.816936447 | 8.12E−158 |
| Krt18 | 0.274491431 | 1.10E−261 |
| Krt222 | 0.379569529 | 0 |
| Krt8 | 0.436565392 | 1.52E−251 |
| Cdh1 | 1.161112119 | 8.78E−130 |
| Epcam | 0.400290103 | 1.93E−167 |
| Cacybp | 0.100023733 | 0.025964511 |
| Ceacam1 | 0.95889773 | 1.79E−220 |
| Zeb2 | 0.528985849 | 1.09E−93 |
| Tgfb1 | 0.522272019 | 7.91E−82 |
| Tgfbr1 | 0.905995223 | 2.37E−211 |
| Cd36 | 1.092748198 | 1.73E−280 |
| Cd44 | 0.923327024 | 6.82E−08 |
| L1cam | 0.895770782 | 4.76E−08 |
| Fn1 | 2.511520987 | 6.64E−128 |
| Lgals3 | 1.131734887 | 1.83E−219 |
| Hba-a1 | 4.014739639 | 2.53E−275 |
| Hba-a2 | 3.96942072 | 0 |
| Hbb-bs | 3.976006038 | 7.47E−131 |
| Hbb-bt | 3.930386043 | 9.22E−211 |

TABLE 4
tCZCs/ntCZC enrichment for metastasis and invasion-associated gene sets

| Description | p value | Genes |
|---|---|---|
| GAC_METS_EXP_UP | 5.37E−08 | S100A10, RPL17, ANXA1, |
| | | S100A11, TAGLN2, VIM, RPS12, |
| | | TPT1, RPL12, S100A6 |
| HUMAN_GASTRIC_PPP1R1B+_ | 6.90E−08 | IER3, RPL23A, RPL17, SOCS3, |
| CELLS | | RPL14, RPS6, RPL12, RPS9 |
| MOUSE_GASTRIC_ | 6.90E−08 | ID1, PLAC8, ANXA1, S100A11, |
| IMMATURE_PIT_CELLS | | TAGLN2, RPS12, RPL12, S100A6 |
| HUMAN_DUODENAL_STEM_ | 2.00971E−06 | IER3, RPL5, RPL14, XIST, RPS6, |
| CELLS | | RPS12, RPL12 |
| >Up-regulated genes in the | 2.48612E−05 | IFITM1, IFITM2, PLAC8, |
| subpopulation of invasive PyMT | | COX4I1, RPL14, RPS6, HEBP1, |
| cells (breast cancer) compared | | VIM, MAP1LC3B, S100A6 |
| to the general population of | | |
| PyMT cells. | | |
| INTESTINAL CRYPT BASED | 2.68587E−05 | RPL23A, RPL14, RPS6, RPS12, |
| COLUMNAR CELL | | RPL12 |
| SIGNATURE 1_SMALL | | |
| INTESTINE_NORMAL_ | | |
| MOUSE | | |
| INTAEPITHELIAL T | 3.3066E−05 | RPL23A, RPL17, RPS6, RPL12, |
| CELLS_SMALL | | RPS9 |
| INTESTINE_NORMAL_ | | |
| MOUSE | | |
| HUMAN_GASTRIC_ | 4.98783E−05 | IL1R2, ID1, PLAC8, S100A11, |
| IMMATURE_PIT_CELLS | | TAGLN2, S100A6 |
| HUMAN_GASTRIC_MATURE_ | 4.98783E−05 | IL1R2, PLAC8, S100A11, |
| PIT_CELLS | | TAGLN2, BASP1, S100A6 |
| BULK_GAC_PRIM | 4.98783E−05 | S100A10, PLAC8, TAGLN2, |
| | | VIM, TPT1, S100A6 |
| HUMAN_DUODENAL_DIFFER | 4.98783E−05 | RPS15, RPL5, RPL14, RPS6, |
| ENTIATING_STEM_CELLS | | RPS12, RPL12 |
| HUMAN_GASTRIC_IMMUNE_ | 4.98783E−05 | IFITM1, IFITM2, ANXA1, |
| CELLS | | ALOX5AP, FKBP5, VIM |
| REVIVAL STEM CELL | 0.000168195 | TSPO, ANXA1, S100A11, |
| SIGNATURE (ENRICHED IN | | RPS25, CD24A, F11R, RPL12, |
| METASTASIS INITIATING | | BASP1 |
| CELLS)_SMALL | | |
| INTESTINE_NORMAL_ | | |
| MOUSE | | |
| ORTHO_IMPLANT_ | 0.000221478 | IFITM1, S100A9, FABP5, IL1R2, |
| shRNANalcn_UP | | WFDC21, S100A8, STFA2L1 |
| HUMAN_GASTRIC_PIT_ | 0.000441998 | LCN2, RPL5, RPL17, ID1, |
| MUCOUS_GLAND_CELL | | SOCS3 |
| MOUSE_GASTRIC_MATURE_ | 0.001035077 | FABP5, ID1, PLAC8, TAGLN2, |
| PIT_CELLS | | S100A6 |
| HUMAN_DUODENAL_ | 0.001035077 | IFITM1, IFITM2, ANXA1, |
| IMMUNE_CELLS | | ALOX5AP, VIM |
| HUMAN_OESOPHAG_LATE | 0.001035077 | S100A9, S100A8, S100A10, |
| SUPRABASAL_CELL | | ANXA1, CXCR2 |
| HUMAN_GASTRIC_ | 0.001035077 | FABP5, IL1R2, S100A10, |
| ISTHMUS_CELLS | | TAGLN2, S100A6 |
| MOUSE_GASTRIC_TUFT_ | 0.001035077 | CD300LF, PLAC8, BPGM, |
| CELLS | | ALOX5AP, CD24A |
| TUFT CELLS_SMALL | 0.001162074 | S100A11, ALOX5AP, CD24A, |
| INTESTINE_NORMAL_ | | S100A6 |
| MOUSE | | |
| >Genes associated with | 0.001623685 | FABP5, IGFBP6, PLAC8, VCAN, |
| migration rate of 40 human | | VIM, S100A6 |
| bladder cancer cells. | | |
| HUMAN_GASTRIC_PIT_ | 0.002383737 | FABP5, PLAC8, ANXA1, |
| MUCOUS_CELL | | S100A11, TAGLN2, S100A6 |
| SSCC SIGNATURE (SINGLE | 0.003367063 | ANXA1, BASP1 |
| PROFILED STEM CELL | | |
| CLUSTER)_SMALL | | |
| INTESTINE_NORMAL_ | | |
| MOUSE | | |
| CD33 + Myeloid | 0.003745456 | CD14, S100A8, CLEC4D, |
| | | DUSP1, TSPO, CD33, CCR1, |
| | | BST1, VCAN, CEBPD |
| Tuft_2 | 0.005471896 | CD300LF, S100A11, HEBP1, |
| | | BASP1 |
| IAC_METS_EXP_UP | 0.007166806 | COX411, COX7A2L, VIM, TPT1, |
| | | S100A6 |
| >Genes mediating | 0.009975629 | CD14, IGFBP6, IER3, TSPO, |
| programmed cell death | | ANXA1 |
| (apoptosis) by activation of | | |
| caspases. | | |
| CD14 + Monocytes | 0.013725105 | CD14, TSPO, CD33, CCR1, |
| | | BST1, VCAN, CEBPD |
| HUMAN_DUODENAL_ | 0.017490082 | RPL5, RPL17, RPS6, RPS12 |
| TRANSIENT_AMPLIFYING_CELLS | | |
| HUMAN_GASTRIC_ | 0.017490082 | FABP5, ID1, TSPO, BASP1 |
| METALLOTHIONEIN_CELLS | | |
| VECCHI_GASTRIC_ | 0.029859694 | IFITM1, LCN2, S100A10, |
| CANCER_EARLY_UP | | IFITM2, S100A11, UPP1, S100A6 |
| ENTERO-ENDOCRINE CELL | 0.031567233 | FABP5, CD24A, VIM |
| PROFILE_SMALL | | |
| INTESTINE_NORMAL_ | | |
| MOUSE | | |
| ENTEROCYTE POPULATION | 0.031567233 | S100A10, PLAC8, S100A11 |
| 5_SMALL | | |
| INTESTINE_NORMAL_ | | |
| MOUSE | | |

To understand the fate of ntCZCs, the inventors looked for ZSG+ cells in the lungs and kidneys of aged V1^{R} and Pdx1^{R}Nalcn^{+/+}, Nalcn^{+/Flx} and/or Nalcn^{Flx/Flx} mice. Remarkably, ZSG⁺ cell clusters were readily detected in these organs in Nalcn deleted animals, but were absent or detected at significantly lower levels, in Nalcn^{+/+} mice; suggesting that ntCZCs traffic to, and embed within, distant organs (FIG. 1d-f; FIG. 2b, c). To test this more directly, the inventors injected separate aliquots of 25,000 ntCZCs isolated from P1^{R}Nalcn^{Flx/Flx} mice into the tail veins of six immunocompromised mice. All recipient mice remained clinically well after an average of 100 days but contained numerous ZSG⁺/Cdh1⁻/Icam1⁺ donor-cell clusters within their lungs, liver, kidneys and peritoneum at a frequency similar to metastatic tumours formed by tail vein injections of tCZCs (FIG. 1f-h; FIG. 2c). Trafficked ntCZCs formed apparently normal structures in target organs, the most extreme example being kidney glomeruli and tubules (FIG. 1f-i). Thus, NALCN regulates cell shedding from solid tissues independent of cancer.

### Discussion

The inventors' data indicate that NALCN is involved in the regulation of stem cell-like epithelial cell shedding. The inventors observed upregulation of genes associated with EMT and invasion within 72 hours of deleting Nalcn from normal gastric stem cells (Table 2). The inventors also observed that blocking NALCN using a pharmaceutical agent causes an increase in the level of stem cell-like cell shedding which equalled the level in non-tumour bearing mice having non-functional NALCN. The movement of epithelial cells to distinct tissues such as the lungs, liver, kidneys are peritoneum and the formation of normal structures in the target organs demonstrate the usefulness of such modified cells in repairing or regenerating organs or tissues.

### Example 2

The inventors conducted two sets of experiments to characterise the potential of non-tumour Circulating ZS-Green labelled Cells (ntCZCs) to traffic to kidneys and embed, potentially enabling renal repair.

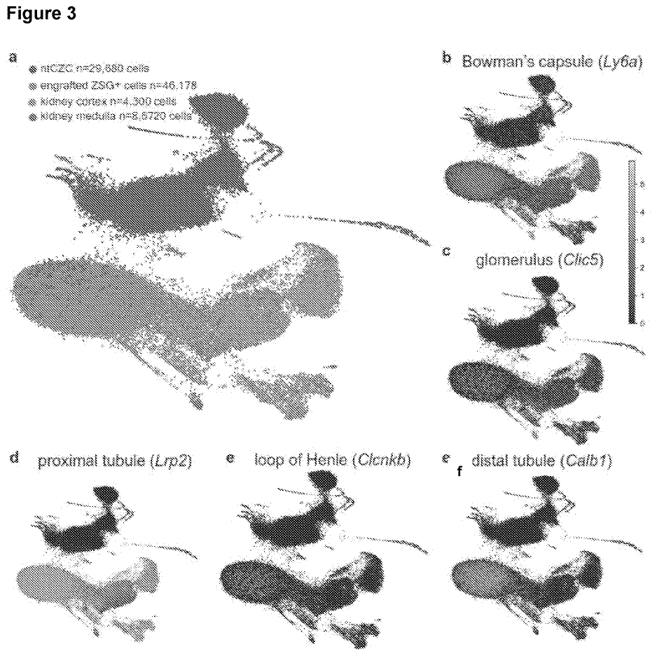

First, donor ntCZCs were isolated from Prom-1; Nalcn^{Flx/Flx} mice and injected into immunocompromised mice (FIG. 1g-i). We isolated ntCZC cells that are ZSG+ cells from the kidneys of these mice and analysed them by single cell RNA sequencing (scRNAseq) analysis. We compared these transcriptomes to transcriptomes of circulating ntCZCs and kidney cells taken from immunocompromised mice (FIG. 3a). The transcriptomes of the trafficked ntCZCs that had embedded within kidneys were distinct from both ntCZC donor cells and kidney cells. Trafficked ntCZCs expressed markers of mature kidney cells including those of renal tubules and glomeruli (FIG. 3b-e).

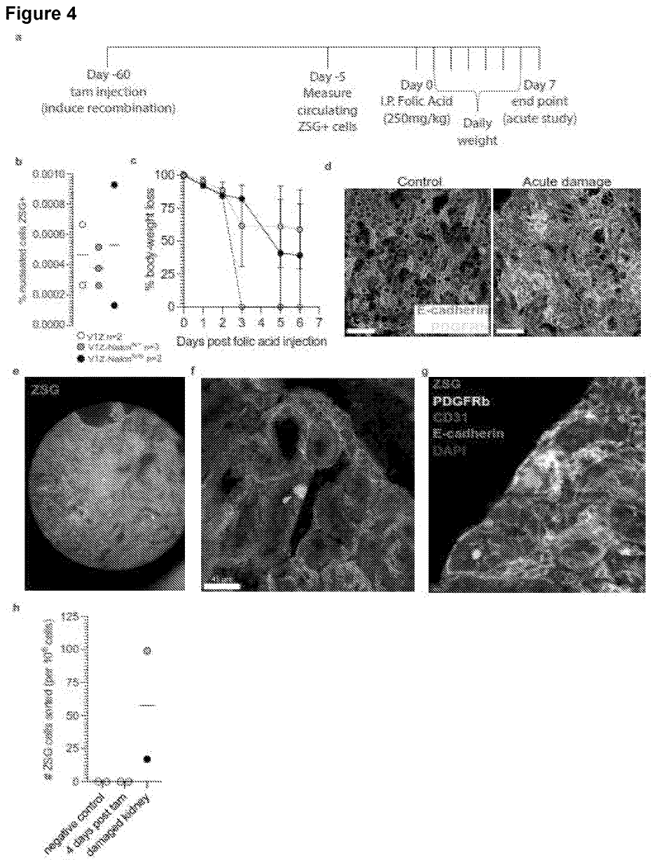

To test if this process operates in whole animal (non-donor) systems in the context of renal damage, we utilized an established model of acute kidney damage in which high dose folic acid crystalizes in the kidney causing inflammation and fibrosis. We conducted these experiments in Villin-1; Nalcn^{+/+} (n=2), Villin-1; Nalcn^{+/Flx} (n=3), and Villin-1; Nalcn^{Flx/Flx} (n=2) mice (FIG. 4a). In this model, Cre-recombination occurs in the intestinal epithelium and is reported using the lineage reporter ZSG. Recombination was induced in intestinal epithelium 60 days prior to kidney damage. Five days prior to folic acid exposure, we confirmed the shedding and circulation of ntCZCs in the peripheral blood of all animals (FIG. 4b). As expected, administration of folic acid caused rapid weight loss: Villin-1; Nalcn^{+/+} mice reached endpoint within three days, while Villin-1; Nalcn^{+/Flx} and Villin-1; Nalcn^{Flx/Flx} mice reached endpoint six days (FIG. 4c). Histological evaluation of the kidneys from animals at end-point confirmed the presence of inflammation and fibrosis (PDGFRb staining) and the presence of trafficked ntCZCs to sites of kidney damage (FIG. 4d-g). We were able to further isolate ZSG+ cells from Villin-1; Nalcn^{+/Flx} and Villin-1 Nalcn^{Flx/Flx} mice (FIG. 4h). These data suggest that ntCZCs can migrate to, and incorporate within, kidneys to adopt a transcriptome of the mature target tissue and contribute to renal function, repairing kidney damage.

1. A modified stem cell-like cell for use in organ regeneration or repair, wherein the modified stem cell-like cell exhibits reduced ion flow through sodium leak channel (NALCN).

2. A ion channel modulator for use in organ regeneration or repair, wherein the ion channel modulator reduces ion flow through sodium leak channel (NALCN).

3. The ion channel modulator for use according to claim 2, wherein the ion channel modulator targets NALCN and/or one of the proteins associated with NALCN, wherein the proteins associated with NALCN are selected from: GPCR (M3 muscarinic receptor M3R, TACR1, CaSR), UNC80, UNC79, FAM155A (NLF1A), Fam155B, SLO2.1

4. The ion channel modulator for use according to claim 2 or 3, wherein the ion channel modulator targets the pore turret domains, voltage sensing domains or linker domains of NALCN.

5. The modified stem cell-like cell or ion channel modulator for use according to any preceding claim wherein the organ to be regenerated is selected from: thymus, adrenal gland, thyroid gland, intestine, lungs, heart, liver, blood vessels, germ cells, nervous system, eye tissues, hair cells, kidney and bladder, skin, hair follicles, pancreas, bone, and cartilage.

6. The modified stem cell-like cell or ion channel modulator for use according to claim 5 for use in the treatment of chronic kidney disease, for example glomerulonephritis and/or renal failure, lung disease, for example COPD, or liver fibrosing diseases.

7. The modified stem cell-like cell or ion channel modulator for use according to any preceding claim, wherein the reduced ion flow through NALCN is transient.

8. A pharmaceutical composition comprising the modified stem cell-like cell of claim 1 or 7 or ion channel modulator of any one of claims 2-4 or 7.

9. A method of repairing or regenerating an organ in a subject, comprising administering a therapeutically effective amount of the modified stem cell-like cell of claim 1 or 7, the ion channel modulator of claim 2-4 or 7 or the composition according to claim 8 to the subject.

10. The method according to claim 9 wherein the method further comprises obtaining stem cell-like cells from the subject to be treated.

11. The method according to claim 10 wherein the method further comprises modifying the stem cell-like cells such that the stem cell-like cell exhibits reduced ion flow through NALCN.

12. The method according to claim 11 wherein the stem cell-like cells are modified by:
contacting a stem cell-like cell with an ion channel modulator, wherein the ion channel modulator inhibits ion flow through NALCN; and/or
introducing a mutation in and/or deleting Nalcn and/or reducing expression of Nalcn in the stem cell-like cell, wherein the mutation and/or deletion and/or reduced expression results in reduced ion flow through NALCN.

13. The modified stem cell-like cell for use according to claim 1 or 7, pharmaceutical composition according to claim 8 or method according to claim 9-12, wherein the modified stem cell-like cell is an epithelial stem cell.

14. The modified stem cell-like cell for use according to claim 1 or 7, pharmaceutical composition according to claim 8 or method according to claim 9-12, wherein the modified stem cell-like cell:
i) comprises a mutation in Nalcn which reduces ion flow through NALCN; or
ii) has a knockout of Nalcn;
iii) exhibits reduced expression of Nalcn; or
iv) has been treated with an ion channel modulator to reduce ion flow through NALCN.

15. The modified stem cell-like cell for use according to claim 14, wherein the reduced ion flow through NALCN is transient.

16. A method of preparing a modified stem cell-like cell the method comprising:
i) contacting a stem cell-like cell with an ion channel modulator, wherein the ion channel modulator inhibits ion flow through NALCN; or
ii) introducing a mutation in and/or deleting Nalcn and/or reducing the expression of Nalcn in a stem cell-like cell, wherein the mutation and/or deletion and/or reduced expression results in reduced ion flow through NALCN.

17. The method according to claim 16 wherein the stem cell-like cell is an epithelial stem cell.

18. The method according to claim 16 or 17 wherein the contacting is performed in vitro or ex vivo.

19. The method according to any one of claims 16-18 wherein the method further comprises expanding the modified stem cell-like cells.

20. A method of increasing the level of solid tissue cell shedding of stem cell-like cells comprising introducing a mutation in and/or deleting Nalcn and/or reducing the expression of Nalcn in a stem cell-like cell, wherein the mutation and/or deletion and/or reduced expression results in reduced ion flow through NALCN.

21. A method of increasing the level of solid tissue cell shedding of stem cell-like cells comprising contacting a cell with an ion channel modulator wherein said ion channel modulator reduces ion flow through NALCN.

22. The method according to claim 21, wherein the cell is contacted in vitro, ex vivo or in vivo.

23. The method according to claim 20 or 21 wherein the solid tissue is epithelial tissue.

24. A kit comprising a modified stem cell-like cell, wherein the modified stem cell-like cell exhibits reduced ion flow through NALCN and optionally instructions for use.

25. A kit according to claim 24 wherein the kit further comprises a NALCN modulator.

26. A kit comprising a means to obtain a cell sample from a subject and components to modify a cell to reduce ion flow through NALCN.

26. A kit according to any one of claim 24 or 25, further comprising components for culture of said modified stem cell-like cell.
